(19) United States
(12) Patent Application Publication
(10) Pub. No.: US 2009/0005253 A1
(43) Pub. Date: Jan. 1, 2009

(54) Double Hybrid System Based on Gene Silencing by Transcriptional Interference

(75) Inventors: Alain Fournier, Chatenay-Malabry (FR); Ralf Behrens, Deutsch Evern (DE); Silvio Bandiera, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA S.A., Antony (FR)

(21) Appl. No.: 11/577,411

(22) PCT Filed: Oct. 24, 2005
(86) PCT No.: PCT/FR2005/002646

§ 371 Date: Feb. 7, 2008

(30) Foreign Application Priority Data
Oct. 25, 2004 (FR) 04/11,372

(51) Int. Cl. C40B 30/00; C12N 1/19; C12N 5/06; C12N 15/00; C12N 5/04; C12Q 1/68

(52) U.S. Cl. 506/7; 435/254.2; 435/325; 435/419; 435/348; 435/254.21; 435/254.22; 435/254.23; 435/6; 435/320.1

(57) ABSTRACT

The invention relates to a novel double hybrid system and to the uses thereof. This system provides, in particular, a tool that enables the detection of the interruption of a protein-protein interaction. The developed system uses transcription interference as a mechanism for detecting a breaking up of interacting protein pairs. The developed double hybrid system can be applied to the screening of molecules enabling the detection of molecules breaking up a protein-protein interaction as well as enabling the identification of alleles lacking in the interaction of proteins involved in the protein-protein interactions.

The present invention relates to a novel double-hybrid system based on gene silencing by transcriptional interference. It likewise relates to the use of this system for the identification of biologically active compounds acting on protein-protein interaction.

The double-hybrid system (Y2H: ‘Yeast Two hybrid system’) has been used extensively in yeast for the identification of protein-protein interactions (cf. especially: Chien, C. T. et al., 1991, Fields, S., and O, Song, 1989, Cagney, G. et al., 2000, 6. Causier, B., and B. Davies, 2002, Coates, P. J., and P. A. Hall. 2003).

The double-hybrid technique rests on the fact that a DNA-binding domain and a transcription activation domain contained in distinct chimeric proteins can activate the transcription of a gene when these two domains are sufficiently near to one another. A currently used version of the double-hybrid system in yeast profits from properties of the GAL4p protein of the yeast Saccharomyces cerevisiae (Fields, S., and O, Song, 1989).

Among others, the protein GAL4p activates the transcription of the GAL1 gene necessary for the utilization of galactose by yeast and is formed of two domains: an N-terminal domain which specifically binds a DNA sequence (UAS_{G} for upstream activated sequence for the yeast GAL genes) and a C-terminal domain containing an acidic region allowing transcriptional activation.

This first double-hybrid system causes three actors to intervene: two fusion proteins and a construct in which a reporter gene is placed under the control of the promoter of the GAL1 gene (Pgal1). A first protein fused to the DNA-binding domain (DBD) of the GAL4p protein is expressed in a yeast S. cerevisiae carrying the construct described above. A second protein fused to the transcription activation domain (AD) of GAL4p is co-expressed in this same yeast. In the case of interaction of the two fusion proteins, the bringing together of the GAL4-AD transcription activation domain and of the DNA-binding domain permits the reconstitution of a functional GAL4p transcription factor, which permits the induction of the expression of the reporter gene placed under the control of Pgal1.

The choice as reporter gene of a gene essential to the viability of the yeast (like, for example, the gene HIS3) allows easy screening of the strains for which the reporter gene is expressed and thus for which an interaction between the two hybrid proteins takes place. The strains for which such an interaction does not take place cannot activate Pgal1 and cannot grow if the reporter gene is a gene essential to the viability of the yeast.

More recently, so-called reverse double-hybrid systems have been developed (rY2H:‘reverse Yeast Two hybrid system’). In these systems, the activation of the reporter gene is induced in the absence of a protein-protein interaction, which is especially useful for the identification of mutant proteins which have lost the capacity to interact with their partner as well as for the identification of molecules specifically inhibiting protein-protein interaction.

Young et al have thus perfected a high-efficiency screening test based on a reverse double-hybrid system in order to identify calcium channel modulators (Young, K. et al., 1998). This rY2H system exploits the properties of the CYH2 gene. The CYH2 gene encodes a ribosomal protein L29, a component of the ribosomal subunit 60S, which confers sensitivity with respect to cycloheximide to cells (Kaufer, N. F. et al., 1983). A reporter cassette Pgal1-CYH2 is introduced into a strain of yeast resistant to cycloheximide having an endogenous mutated CYH2 gene. The allele CYH2 of wild-type being dominant, the growth of the reporter cell is inhibited on a medium containing cycloheximide when the interaction between the two proteins occurs (and thus when the reporter cassette is transcribed). Interruption of the interaction attenuates the toxic effect of cycloheximide and allows the reporter cell to grow on a medium containing cycloheximide (Leanna, C. A., and M. Hannink. 1996).

However, problems associated with the stability of the mRNA and of the protein as well as differences at the level of the activity of wild-type and mutated CYH2p proteins make this system complex to use and not sensitive enough for the detection of dissociation of protein-protein interactions (reverse Y2H interactions).

This system additionally has the disadvantage of being relatively difficult to use in order to obtain maximum sensitivity because its response with respect to reverse Y2H interactions depends on several molecules. Thus the competition between wild-type and mutant CYH2p at the level of incorporation in the ribosomes affects the sensitivity of the reporter. In addition, fluctuations at the level of the capture of cycloheximide and of its reactivity on the ribosomes very probably influence the response capacity of the system.

Another reverse double-hybrid system, based on the use of the reporter gene URA3 in combination with its protoxic substrate: 5-fluoroorotic acid (5-FOA) has likewise been described (Huang, J., and S. L. Schreiber. 1997, Vidal, M., et al., 1996, WO9632503). In this system, the induction of the expression of the protein URA3p inhibits cell growth because the 5-FOA is then transformed into its toxic analog: 5-fluoro-UTP. On the contrary, URA3 inactivation allows cell growth because the 5-FOA is then no longer metabolized and the cells use the uracil supplied in the medium. This system is used in order to identify mutant proteins which have lost their capacity to interact with their normal partner (Burke, T. W. et al., 2001; Daros, J. A. et al., 1999; Puthalakath, H., et al., 1999).

The configuration and the mechanism of this system are very similar to those of the reverse double-hybrid system described above (Young, K. et al., 1998). Consequently, this system has the drawbacks raised above. In addition, the system based on the use of 5-FOA has the disadvantage of generating false positives connected with the system itself. In fact, the cell growth is connected with the inactivation of the wild-type URA3. Consequently, compounds inhibiting the URA3p protein allow cell growth. These compounds thus generate a false-positive signal during the use of the system for the identification of molecules specifically inhibiting the protein-protein interaction.

This system likewise has the disadvantage of the use of 5-FOA which is a relatively expensive chemical agent.

Finally, a relay system comprising a cascade of two reporter genes and named ‘Split-Hybrid’ has likewise been described (WO9526400 and WO9813502). This rY2H system has been used in order to identify mutations in the protein CREB which interrupt its combination with CBP (Crispino, J. D. et al., 1999). In these systems, a double-hybrid interaction activates the expression of a first reporter protein which, in its turn, controls the expression of a second reporter gene used for the growth selection. This system, however, is complex to use and not sensitive enough for the detection of reverse Y2H interactions.

The systems described above thus depend on the activity of several molecules (two reporter genes, a reporter gene and a toxic substance, a wild-type and a mutated reporter gene). This increases the complexity of employing them especially when maximum sensitivity is sought. It would be desirable to have a system which is both simpler and more economical, while having good sensitivity.

### GENERAL DESCRIPTION OF THE INVENTION

The present invention relates to a novel double-hybrid system based on the silencing of genes by transcriptional interference. The double-hybrid system according to the present invention is simple, does not require addition of toxic substances to the medium and can be used without integration into the genome being necessary.

A first subject of the invention relates to a cell containing an interference DNA construct, said construct comprising:

A reporter gene placed under the control of a first promoter,

One or more inducible promoter(s), so-called interference promoter(s), chosen and positioned so that its (their) activation involves a transcriptional interference of the first promoter, leading to a detectable decrease in the expression of the reporter gene,

said cell additionally expressing:

A first chimeric protein (Y-AD) formed of a transcription activation domain (AD) fused to a protein Y capable of interacting with a partner protein X,

A second chimeric protein (X-DBD) formed of a first DNA-binding domain (DBD) fused to a second domain formed by a protein X capable of interacting with the protein Y, the interaction of the two chimeric proteins X-DBD and Y-AD leading to the formation of a functional transcription factor activating the interference promoter(s).

According to one embodiment, the present invention relates to a cell such as described above whose promoter regulating the expression of the reporter gene is an inducible promoter, whose protein Y is capable of interacting with two partner proteins X and Z, the cell additionally expressing a third chimeric protein (Z-DBD) formed of a DNA-binding domain (DBD), fused to a second domain formed by a protein Z capable of interacting with the protein Y, the interaction of the two chimeric proteins Z-DBD and Y-AD leading to the formation of a functional transcription factor activating the expression of the reporter gene.

In a preferred embodiment, the present invention relates to a cell such as defined above whose inducible promoter regulating the expression of the reporter gene comprises a sequence capable of interacting with the DNA-binding domain (DBD) of the chimeric protein (Z-DBD).

The invention likewise relates to a cell such as defined above whose promoter regulating the expression of the reporter gene is a constitutive promoter.

The invention likewise relates to a cell such as described above, which is a host cell transformed or transfected by the interference DNA construct and by DNA constructs coding for the chimeric proteins, the whole of these constructs being carried by one or more nonintegrative vectors.

Another aspect of the invention relates to a cell such as defined above, which is a host cell transformed or transfected by DNA constructs coding for the chimeric proteins, these constructs being carried by one or more nonintegrative vectors, and wherein the interference DNA construct is integrated into the genome of the cell.

The invention likewise relates to a cell such as defined above whose interference DNA construct and the DNA constructs coding for the chimeric proteins are integrated into the genome of the cell.

In a preferred embodiment, the present invention relates to a cell such as described above whose interference DNA construct is integrated into a locus free of perturbatory genomic transcription activities.

The invention likewise relates to a cell such as defined above, which is chosen from the group formed by the cells of mammals, of insects, of plants and of yeasts.

The invention likewise relates to a cell such as described above, wherein yeast cells are concerned.

In a preferred embodiment, the invention relates to a cell such as defined above, wherein yeasts of the species Saccharomyces cerevisiae, Schizosaccharomyces pombe, Kluyveromyces lactis, Pichia pastoris, Saccharomyces carlsbergensis or Candida albicans are concerned.

The invention also relates to a cell such as defined above with at least one interference promoter positioned downstream of the reporter gene and of the first promoter and in an orientation opposite to the latter (DI).

The invention also relates to a cell such as described above with at least one interference promoter positioned downstream of the reporter gene and of the first promoter and in the same orientation as the latter (nDI).

The invention likewise relates to a cell such as defined above with at least one interference promoter positioned upstream of the reporter gene and of the first promoter and in the same orientation as the latter (UI).

Another aspect of the invention relates to a cell such as described above with at least one interference promoter positioned on both sides of the first promoter and of the reporter gene, the interference promoter(s) situated downstream of the first promoter and of the reporter gene having a convergent orientation with respect to the first promoter and the interference promoter(s) positioned upstream of the first promoter and of the reporter gene having an orientation identical to that of the first promoter (UDI).

The invention additionally relates to a cell such as defined above with at least one interference promoter positioned on both sides of the first promoter and of the reporter gene, the interference promoter(s) situated downstream of the first promoter and of the reporter gene having a paired orientation with respect to the first promoter and the interference promoter(s) positioned upstream of the first promoter and of the reporter gene having an orientation identical to that of the first promoter (nUDI).

In a preferred embodiment, the invention relates to a cell such as defined above and whose reporter gene is a gene essential to the survival of the cell.

The invention likewise relates to a cell such as described above and whose reporter gene is a gene indispensable to the primary metabolism, to cell division, to protein synthesis, to DNA synthesis or RNA synthesis.

The invention likewise relates to a cell such as defined above whose reporter gene is not in itself alone essential to the survival of the cell, but is essential to the survival of the cell when its transcription is inhibited in association with one or more reporter genes of the same type, the expression of which is or is not controlled by the transcriptional interference system.

In a preferred embodiment, the invention relates to a cell such as described above whose inducible interference promoter(s) comprise a sequence capable of interacting with the DNA-binding domain (DBD) of the chimeric protein (X-DBD).

The invention likewise relates to a cell such as defined above whose inducible interference promoter(s) comprise a sequence capable of interacting with a protein having a DNA-binding domain (DBD) chosen from the group formed by: GAL4 UAS, LexAop, ciop and TetRop and such that the DNA-binding domain of the chimeric protein X-DBD is the corresponding DBD (respectively GAL4, LexA, cI or TetR).

Another aspect of the invention relates to a cell such as described above whose transcription activation domain (AD) of the chimeric protein Y-AD is chosen from the group formed by the transcription activation domains of the following proteins: B42, VP16 and GAL4p.

In a preferred embodiment, the invention relates to a cell such as defined above whose interference DNA construct is bordered at its ends by one or more unidirectional or bidirectional transcription terminators.

Another aspect of the invention relates to a process for identification of a compound inhibiting the interaction of a first protein X with a second protein Y, comprising the following steps:
a) culture cells such as defined above,
b) incubate said cells in the presence of the compound to be tested,
c) compare the expression of the reporter gene in the presence and in the absence of said compound, an increase in the expression of the reporter gene being the indication that the compound to be tested is an inhibitor of the interaction of the protein X with the partner protein Y expressed by the cultured cells.

The invention additionally relates to the use of cells such as defined above for the identification of compounds inhibiting protein-protein interaction.

The invention likewise relates to the use of cells such as described above for the screening of cDNA banks or banks of peptides in order to identify peptides or protein factors specifically abrogating a protein-protein interaction.

Another aspect of the invention relates to a kit for the setting up of a double-hybrid system comprising:

A first DNA construct comprising:
A reporter gene placed under the control of a first promoter,
One or more inducible promoters, chosen and positioned so that its (their) activation involves a transcriptional interference of the first promoter, leading to a detectable decrease in the expression of the reporter gene,

A second DNA construct coding for:
A first chimeric protein (Y-AD) formed of a transcription activation domain (AD) fused to a partner protein Y capable of interacting with a partner protein X,

A third DNA construct coding for:
A second chimeric protein (X-DBD) formed of a first DNA-binding domain (DBD) fused to a second domain formed by a protein X capable of interacting with the protein Y, the interaction of the two chimeric proteins X-DBD and Y-AD leading to the formation of a functional transcription factor activating the interference promoter(s) when the two chimeric proteins are expressed in a host cell.

The invention likewise relates to a kit such as defined above, wherein the promoter regulating the expression of the reporter gene is an inducible promoter, wherein the protein Y is capable of interacting with two partner proteins X and Z, and wherein said kit comprises a fourth DNA construct coding for a third chimeric protein (Z-DBD) formed of a DNA-binding domain (DBD) fused to a second domain formed by a protein Z capable of interacting with the protein Y, the interaction of the two chimeric proteins Z-DBD and Y-AD leading to the formation of a functional transcription factor activating the expression of the reporter gene.

Another aspect of the invention relates to a process for identification of a compound inhibiting the interaction of a first protein X with a second protein Y, but not inhibiting or inhibiting less the interaction between the protein Y and a third protein Z, comprising the following steps:
a) culture cells such as defined above,
b) incubate said cells in the presence of the compound to be tested,
c) compare the expression of the reporter gene in the presence and in the absence of said compound, an increase in the expression of the reporter gene being the indication that the compound to be tested is an inhibitor of the interaction of the protein X with the partner protein Y, but that this product does not inhibit or inhibits less the interaction between the protein Y and the protein Z.

The invention finally relates to a yeast integration vector containing two fragments homologous to the upstream and downstream regions of the open reading frame of the gene URA3 of S. cerevisiae and allowing integration by homologous recombination at the level of the locus URA3 of a sequence inserted between these two fragments.

### DETAILED DESCRIPTION OF THE INVENTION

The present invention relates to a novel double-hybrid system based on the silencing of genes by transcriptional interference. The double-hybrid system according to the present invention is simple, does not require addition of toxic substances to the medium and can be used without integration into the genome being necessary.

Transcriptional interference is defined as the perturbation of the activity of a first promoter when a second promoter is activated. Thus, the transcription activity of a promoter reduces a transcription initiated at the level of another promoter. Transcription interference has been described, for example, in yeast (Greger, I. H., and N. J. Proudfoot. 1998, Springer, Col. et al., 1997, Peterson, J. A., and A. M. Myers, 1993, Puig, S. et al., 1999, Martens et al., 2004).

A first subject of the invention relates to cells carrying one or more constructs allowing the expression of a reporter gene in the absence of a particular protein-protein interaction, although when this interaction takes place, a detectable decrease in the expression of said reporter gene is noted.

More particularly, the cells according to the present invention contain one or more DNA constructs, so-called interference constructs, said construct(s) comprising:

A reporter gene placed under the control of a first promoter,

One or more inducible promoter(s), so-called interference promoters, chosen and positioned so that its (their) activation involves a transcriptional interference of the first promoter(s), leading to a detectable decrease in the expression of the reporter gene, said cell additionally expressing:
A first chimeric protein (Y-AD) formed of a transcription activation domain (AD) fused to a protein Y capable of interacting with a partner protein X,
A second chimeric protein (X-DBD) formed of a first DNA-binding domain (DBD) fused to a second domain formed by a protein X capable of interacting with the protein Y, the interaction of the two chimeric proteins X-DBD and Y-AD leading to the formation of a functional transcription factor activating the interference promoter(s).

In a first embodiment, the cells according to the present invention are host cells transformed or transfected by the DNA construct comprising the reporter gene (interference construct) and they are likewise transformed or transfected by two DNA constructs coding for each of the two chimeric proteins. The three constructs can be carried by the same DNA vector or are found on two or three different vectors. All the combinations are conceivable: the three constructs on the same vector, the three constructs on three different vectors, the two constructs coding for the two chimeric proteins on the same vector whereas the third construct is found on a second vector, a construct coding for one of the two chimeric proteins and interference construct on the same vector whereas the construct coding for the second chimeric protein found on a second vector. In this first embodiment, the vectors are nonintegrative vectors.

In a second embodiment of the invention, the interference construct is integrated into the genome of the cells and the latter are transformed or transfected by two DNA constructs coding for each of the two chimeric proteins. The DNA constructs coding for the chimeric proteins can be carried by the same DNA vector or are found on two different vectors, this or these vectors being nonintegrative vectors.

In a third embodiment of the invention, the interference construct as well as the DNA constructs coding for each of the two chimeric proteins are integrated into the genome of the cells. It is likewise possible that only one of the two constructs coding for one of the two chimeric proteins is integrated into the genome and that the second construct is carried by a nonintegrative vector. Finally, in a last embodiment, the interference construct and one of the two constructs coding for one of the two chimeric proteins are carried by nonintegrative vectors although the construct coding for the second chimeric protein is integrated into the genome of the cells.

A reporter gene is customarily defined as a gene coding for an easily detectable product. In a preferential embodiment, the reporter gene used in the context of the present invention is a gene whose expression is essential to the survival of the cell. It can be, for example, a gene indispensable to the primary metabolism, to cell division, to protein synthesis (like the ribosomes), to DNA synthesis or RNA synthesis etc. . . . .

By way of example of this type of gene it is possible to cite all the genes described as essential for the survival of yeast, that is to say approximately ⅙ of the genes which form its genome (Winzeler E. A et al., 1999).

Thus, it can be a gene involved in the primary metabolism and indispensable to the latter or a gene indispensable to cell division. More particularly, it can be a gene coding for an enzyme involved in the biosynthesis of an essential metabolite which can be added or removed from the culture medium.

By way of example of this type of gene it is possible to cite the genes HIS3, URA3, LEU2, LYS2, TRP1, ADE2, MET15 and ARG4 widely used in the yeast S. cerevisiae because easily complementable to the amino acid or to the corresponding nucleic acid base in the culture medium of the yeasts. Other markers based on metabolism can be used; it is possible, by way of example, to cite the gene HIS5 (genes of the yeast S. pombe), the genes URA3 and LEU2 (genes the yeast K. lactis), URA3 (gene the yeast C. albicans), LEU2 (gene of the yeast A. gossypii).

It can likewise be genes involved in the primary metabolism and nonessential to the latter, or genes nonessential to cell division when used alone. On the contrary, if their transcription is inhibited in association with one or more reporter genes of the same type, the combination of the effects can lead to the inhibition of a function essential to the survival of the yeast. It can be, for example, an assembly of genes whose associated roles lead to a function indispensable to the primary metabolism, to cell division, to protein synthesis (like the ribosomes), to DNA synthesis or RNA synthesis etc. . . . .

By way of example of this type of gene it is possible to cite all the genes described as nonessential for the survival of the yeast (that is to say approximately ⅚ of the genes which form its genome (Winzeler E. A et al., 1999)) which in association with one or more genes of the same type then become essential.

However, other reporter genes can likewise be used in the context of the present invention. It is possible to cite, by way of example, the gene coding for the enzyme CAT (chloramphenicol acetyltransferase), the gene luc coding for luciferase, the genes coding for a fluorescent protein like GFP (for ‘Green Fluorescent Protein’), CFP (for ‘Cyan Fluorescent Protein’), YFP (for ‘Yellow Fluorescent Protein’) or RFP (‘Red Fluorescent Protein’), beta-galactosidase, beta-lactamase, beta-glucuronidase.

It can likewise be a marker conferring resistance to a toxic product: the gene kan of the transposon Tn903 (conferring resistance to geneticin (G418)), the gene ble of the transposon Tn5 (conferring resistance to phleomycin), the gene CYHr (conferring resistance to cycloheximide), pat (gene of S. viridochromogenes conferring resistance to bialaphos), nat1 (gene of S. noursei conferring resistance to nurseothricin), hph (gene of E. coli, conferring resistance to hygromycin B). All these genes are functional, especially in yeast (Gutherie and Fink, 2002).

The reporter gene is placed under the control of a first promoter. In a preferred embodiment, this first promoter is a constitutive promoter. A constitutive promoter, as is generally accepted, is a promoter allowing expression relatively independent of the surrounding conditions.

By way of example, in yeast the reporter gene can be expressed under the control of the constitutive mutant promoter ADH1(700) (promoter of the mutated gene ADH1 (Padh1)) (Ruohonen et al., 1995). However, other constitutive promoters can likewise be used in yeast in the context of the present invention. It is possible to cite, by way of example, the promoter TPI (Alber and Kawasaki, 1982), TEF1, TDH3, KEX2 (Nacken et al., 1996) and ACT1 (Ernst, 1986).

As far as the cells of mammals are concerned, it is possible to cite, for example, the promoter CMV (for human cytomegalovirus (CMV) immediate-early enhancer/promoter) (Foecking and Hofstetter, 1986) (Kronman et al., 1992), the promoter EF-1 a (Mizushima and Nagata, 1990), the promoter SV40 (for Simian Virus 40 early enhancer/promoter) (Das et al., 1985), the promoter UB (promoter of the human gene of ubiquitin C (hubC)) (Nenoi et al., 1996; Schorpp et al., 1996), the promoter RSV LTR (for Rous Sarcoma Virus Long Terminal Repeat) of the Rous sarcoma virus (Yamamoto et al., 1980).

The promoters, so-called ‘interference promoter’, are inducible promoters. The latter are chosen and positioned so that their activation involves a transcriptional interference of the first promoter, leading to a detectable decrease in the expression of the reporter gene.

The interference promoters which can be employed in the context of the present invention are inducible promoters whose activation is induced by a transcription factor interacting via a DNA-binding domain (DBD) with a sequence of said promoter (DNA sequence linked by DBD), said transcription factor being capable of initiating the transcription governed by the promoter. Thus the interference promoters which can be employed in the context of the present invention are all the promoters suitable for the setting up of a double-hybrid system in the organism considered.

Preferably, the interference promoters according to the present invention comprise a sequence capable of interacting with a protein having a DNA-binding domain (DBD) (DNA sequence bound by the DBD (UAS)). More particularly, the inducible interference promoters comprise a sequence capable of interacting with the DNA-binding domain (DBD) of the chimeric protein (X-DBD).

These are, for example, the promoters Pgal1, Pgal10, Pgal5, Pgal80, Pmel1, Pgal2, Pgal7, Pgal3, Pgcy1, Plth1, Ppcl10 and Pfur4 (Ren et al., 2000; Svetlov and Cooper, 1995), inducible by the transcription factor GAL4p (in a wild-type cell, the transcription factor GAL4p is especially involved in the expression of genes induced by galactose). Preferably, the inducible promoter employed is the promoter Pgal1.

However, other embodiments of the invention using other inducible expression systems likewise come into the scope of the present invention. Table 1 shows quite a number of inducible systems already used in double-hybrid systems. The inducible promoters of these systems described in the literature are likewise interference promoters in the sense of the present invention. In order to obtain a functional double-hybrid system, all the combinations possible between the DBDs and the ADs in table 1 are conceivable.

TABLE 1

| DNA | DNA- | | |
|---|---|---|---|
| sequence bound | binding | Transcription | |
| by the DBD | domain | activation | Bibliographic |
| (UAS, operator) | (DBD) | domain (AD) | reference |
| LexAop | LexA | B42 | (Gyuris et al., |
| | | | 1993) |
| LexAop | LexA | VP16 | (Vojtek and |
| | | | Hollenberg, 1995) |
| GAL4 UAS | GAL4 | GAL4 | (Elledge and |
| | | | Spottswood, 1991; |
| | | | Fields and Song, |
| | | | 1989) |
| cIop | cI | B42 | (Serebriiskii et |
| | | | al., 1999) |
| TetRop | TetR | B42 | (Xu et al., 1997) |

The inducible interference promoter(s) are positioned so that their activation involves a transcriptional interference of the first promoter, leading to a detectable decrease in the expression of the reporter gene.

The interference promoter(s) can thus be positioned downstream of the first promoter and of the reporter gene; it is then a question of downstream interference because the activation of the interference promoter situated downstream interferes with the activity of the first promoter situated further upstream.

In the context of downstream interference, the interference promoter can, however, have two possible orientations. A first orientation corresponds to a convergent orientation with respect to the first promoter; this configuration has been called downstream interference (or DI). The interference promoter can likewise have a paired orientation with respect to the first promoter; this second configuration has been called nonsense downstream interference (or nDI) which likewise shows an inhibition of the expression of the reporter gene (cf. the example section).

The interference promoter(s) can likewise be positioned upstream of the first promoter and of the reporter gene; it is then a question of upstream interference because the activation of the interference promoter situated upstream interferes with the activity of the first promoter situated further downstream. The interference promoter then has an orientation identical to the first promoter (the two promoters thus have a paired configuration); this configuration has been called upstream interference (or UI).

Another possible configuration is the combination of the two configurations described above (UI and DI system). In this case, at least one inducible interference promoter is positioned on both sides of the first promoter and of the reporter gene. It is then a question of upstream-downstream interference because the activation of the interference promoter(s) situated upstream interfere(s) with the activity of the first promoter situated further downstream and the activation of the interference promoter(s) situated downstream interfere(s) with the activity of the first promoter situated further upstream.

The interference promoter(s) situated downstream of the first promoter and of the reporter gene have a convergent orientation with respect to the first promoter (cf. the DI system above). The interference promoter(s) positioned upstream of the first promoter and of the reporter gene have an orientation identical to that of the first promoter (the two promoters thus have a paired configuration) (cf. UI system above).

This configuration has been called upstream-downstream interference (or UDI).

Another possible configuration is the combination of two configurations described above (UI and nDI system). In this case, at least one inducible interference promoter is positioned on both sides of the first promoter and of the reporter gene. It is then a question of upstream-downstream interference because the activation of the interference promoter(s) situated upstream interfere(s) with the activity of the first promoter situated further downstream and the activation of the interference promoter(s) situated downstream interfere(s) with the activity of the first promoter situated further upstream.

The interference promoter(s) situated downstream of the first promoter and of the reporter gene have a paired orientation with respect to the first promoter (cf. the nDI system above). The interference promoter(s) positioned upstream of the first promoter and of the reporter gene have an orientation identical to that of the first promoter (cf. UI system above), the three promoters thus have a paired configuration.

This configuration has been called nonsense upstream-downstream interference (or nUDI).

In the case of a construct of UI type, an activation of the interference promoter will lead to a reduction in the activity of the first promoter because of transcription interference. The activity of the upstream interference promoter could, however, again allow a sense transcript of the reporter gene to be produced.

In order to prevent the translation of this functional sense transcript, a short open reading frame followed by one or more (for example 2 or 3) stop codons is preferentially inserted between the interference promoter and the first promoter. Transcription interference in a UI system then produces a bicistronic messenger starting from which only the first short open reading frame is translated.

This is likewise the case in a configuration of UDI and nUDI type. In fact, in the case of a construct of UDI or nUDI type, an activation of the interference promoter(s) situated upstream of the first promoter will lead to a reduction in the activity of the first promoter by reason of transcription interference. The activity of the upstream interference promoter could, however, again allow a sense transcript of the reporter gene to be produced.

In order to prevent the translation of this functional sense transcript, a short open reading frame followed by one or more stop codons is preferentially inserted between the interference promoter and the first promoter. Transcription interference in a UDI or nUDI system then produces a bicistronic messenger starting from which only the first short open reading frame is translated.

Preferentially, the interference constructs described above are bordered at their ends by bidirectional transcription terminators. Any functional and bidirectional transcription terminator can be used in order to border the constructs according to the invention. By way of example, it is possible to use the transcription terminator of the gene CYC1 (Tcyc1) (described by Osborne, B. I., and L. Guarente., 1989) as well as the terminator of the gene ADH1 (Tadh1) (described by Irniger, S. et al., 1991).

The bidirectional transcription terminators stop the transcription in a bidirectional manner. This prevents the extension of transcription directed by the first promoter or the interference promoter(s) beyond the construct and likewise protects the system from the possible influence of transcription activities situated outside of the construct.

With the same aim, it is likewise possible to combine unidirectional terminators in a configuration which blocks the transcription in the two senses (by combining two unidirectional terminators blocking transcription in a divergent or convergent orientation in order to obtain a bidirectional terminator). By way of example of unidirectional terminators, it is possible to cite the following terminators: Tpgk1 (Picard et al., 1990), Ttef (from A. gossypii) (Steiner and Philippsen, 1994), This3 (from S. kluyveri) (Weinstock and Strathern, 1993).

The interference promoter according to the present invention is an inducible promoter and is chosen and positioned so that its activation involves a transcriptional interference of the first promoter, leading to a detectable decrease in the expression of the reporter gene.

The inducible promoter is conditionally activated. In the context of the present invention, it is activated by a transcription factor when this is active. According to the present invention, the transcription factor activating the inducible promoter is formed by an assembly of two chimeric proteins (X-DBD and Y-AD) reconstituting an active transcription factor capable of activating the inducible promoter when an interaction takes place between the two chimeric proteins X-DBD and Y-AD.

As has been specified above, the cells according to the present invention express two chimeric proteins:
A first chimeric protein (Y-AD) formed of a transcription activation domain (AD) fused to a partner protein Y, capable of interacting with a partner protein X,

A second chimeric protein (X-DBD) formed of a first DNA-binding domain (DBD) fused to a second domain formed by the protein X, capable of interacting with the protein Y, the interaction of the two chimeric proteins X-DBD and Y-AD resulting in the formation of a functional transcription factor activating the interference promoter(s).

The DNA-binding domain (DBD) of the second chimeric protein X-DBD is thus chosen as a function of the inducible promoter so as to link the DNA at the level of said promoter. Preferably, the DNA-binding domain (DBD) of this chimeric protein is chosen in order to interact with a sequence present in said promoter (DNA sequence linked by the DBD (UAS) (upstream activating sequence)).

The transcription activation domain of the first chimeric protein Y-AD allows the transcription of the inducible promoter in the cell to be activated. Thus, the interference promoter(s) (inducible promoters) are induced by the functional transcription factor formed by the assembly of the two chimeric proteins Y-AD and X-DBD (interacting between them), this functional transcription factor interacting via its DNA-binding domain (DBD) with a sequence of the promoter and being capable of initiating the transcription governed by the promoter via its transcription activation domain (AD).

This constitutes the very principle of the double-hybrid technique and the person skilled in the art is able to choose from multiple promoter/transcription factor pairs allowing the implementation of the present invention.

A currently used version of the double-hybrid system in yeast profits from the properties of the protein GAL4p of the yeast Saccharomyces cerevisiae (Fields, S., and O, Song, 1989) and of the promoter Pgal1. However, the other double-hybrid systems described, especially those described in table 1 can likewise be used in the context of the present invention.

The partner proteins X and Y fused to the DNA-binding domain (DBD) and to the transcription activation domain (AD) respectively can be defined as any pair of proteins which, fused to the DBD and AD respectively, interact in the cell (even if that is not the case in their organisms of origin) in order to result in the formation of a functional transcription factor (X-DBD/Y-AD) activating the inducible promoter(s) such as described above. It can be a question of complete proteins or of fragments of the latter. Thus, it is possible in the context of the present invention to use chimeric proteins generating a double-hybrid signal. That is to say the chimeric proteins whose two partner proteins (or fragment of the partner proteins) interact between them with a sufficient affinity in the cell used, in order to form a functional transcription factor activating the inducible promoter(s), said transcription factor binding the DNA via the binding domain to of the first chimeric protein and activating the promoter(s) by the transcription activation domain (AD) of the second chimeric protein. The cells according to the invention can carry the DNA construct and the constructs coding for the chimeric proteins either integrated or on a nonintegrated vector. It has been confirmed that the best results are obtained with an integrated interference DNA construct.

Preferentially, the interference DNA construct is integrated into the genome of the host cell. The interference constructs are advantageously targeted towards a locus free of potentially perturbatory genomic transcription activities, in order to protect the interference systems from the possible influence of transcriptional activities initiated outside of the reporter cassette.

In order to do that, a novel integration vector of yeast pRB2 has been constructed. This integration vector targets the interference constructs towards a locus free of potentially perturbatory genomic transcription activities (cf. FIG. 3).

It has likewise been shown that the interference systems according to the present invention on a nonintegrated vector are genetically stable, which renders unnecessary the construction of strains having integrated reporter cassettes. This characteristic is very interesting for medicament screening since it is thus possible to easily evaluate numerous strains having different mutations in order to increase the permeability of small molecules.

The cells which can be used for the implementation of the double-hybrid system which is the subject of the present invention consist of any cell capable of expressing the required chimeric proteins in said double-hybrid system described above and of being able to be transformed or transfected by/with the interference DNA construct described above. These host cells are eukaryotic or prokaryotic hosts; they can be cells of mammals, of insects, of plants or more preferentially of yeasts.

In a preferred embodiment, the cells according to the present invention are chosen from the kingdom of the Fungi. Preferentially, the organisms of the kingdom of the Fungi are chosen from the phylum of the Ascomycetes, more preferably, they are chosen from the subphylum of the Saccharomycotina, even more preferably, they are chosen from the class of the Saccharomycetes or of the Schizosaccharomycetes, even more preferably there are chosen from the order of the Saccharomycetales or of the Schizosaccharomycetales, even more preferably there are chosen from the family of the Saccharomycetaceae or of the Schizosaccharomycetaceae, even more preferably they are chosen from the genus Saccharomyces or Schizosaccharomyces; entirely preferably the organisms of the kingdom of the Fungi according to the invention belong to the species Saccharomyces cerevisiae or Schizosaccharomyces pombe. The yeasts of the species Kluyveromyces lactis, Pichia pastoris, Saccharomyces carlsbergensis or Candida albicans are likewise cells coming within the scope of the present invention.

Contrary to the systems described up to now, the double-hybrid systems according to the present invention do not necessitate the addition of toxic or nontoxic substances. This is an advantage because they probably have additional effects on cell physiology and they can interfere with the detection of certain reverse Y2H interactions. The use of a toxic or nontoxic substance, however, remains possible. It can, for example, be interesting to use a substance inhibiting the product of the reporter gene in the case of the use of a reporter gene indispensable to survival, for example of a gene indispensable to the primary metabolism, to cell division, to protein synthesis (like the ribosomes), to DNA synthesis or RNA synthesis etc. . . . . In fact, if the reporter gene is still sufficiently expressed despite the transcription interference and if a total absence of growth of the cells is sought, a substance inhibiting the product of such a reporter gene can be used in order to increase the sensitivity of the double-hybrid system. Thus, for example, if the reporter gene is the gene HIS3, the cell growth of strains of interference yeast (for example S. cerevisiae) can be evaluated on medium depleted in histidine. A competitive inhibitor of the protein HIS3p (3-amino-1,2,4-triazole (3-AT)) can possibly be added to the culture medium in order to observe differences in the level of the level of activity of the reporter gene HIS3. This can allow a greater sensitivity to be achieved and the system to be calibrated (cf example 6).

If, however, the reporter gene is still sufficiently expressed despite the transcription interference and if a total absence of growth of the cells is sought, one possibility is to use in combination other reporter genes which are or are not controlled by the interference systems described above (UI, DI, nDI, UDI and nUDI) which have the effect of increasing the sensitivity of the double-hybrid system. The even partial inhibition of the expression of these reporter genes leads, by addition of the effects, to a complete inhibition of growth. Thus, for example, if one of the reporter genes is the gene HIS3 and one of the others is the gene URA3, both of them controlled by the interference system, the growth of the interference yeast (for example S. cerevisiae) can be evaluated on medium depleted in histidine and in uracil. Although on a medium depleted uniquely in histidine or in uracil it would still be possible to have growth of the yeast having a partial transcription interference of the reporter genes, on a medium depleted in both uracil and in histidine the growth can be completely abolished.

The double-hybrid system according to the present invention can have numerous applications. Thus, it is particularly appropriate for the screening of molecules having a protein-protein interaction inhibition activity.

The identification of protein-protein interactions involved in the pathologies is highly interesting since these interactions provide potential targets for the development of novel medicaments. Once a protein-protein interaction involved in a disease has been identified, it is often very interesting to have available synthetic or natural molecules, specifically dissociating this protein-protein interaction in order on the one hand to validate the pertinence of this interaction in complex biological systems and to have available candidate molecules for the development of a medicament (cf. Vidal, M., and H. Endoh, 1999).

The double-hybrid systems according to the present invention provide simple genetic systems for inhibitor screening of a given protein-protein interaction. Thus in the double-hybrid systems according to the present invention, the molecules dissociating the protein-protein interaction cause the expression of the reporter gene, which allows their identification.

It can be pertinent to choose a reporter gene whose expression is essential to the survival of the cell. Thus, in allowing a re-expression of this essential gene, the molecules inhibiting the protein-protein interaction can consequently be easily identified by following the cell growth.

Thus, the inhibitor screening of the sought protein-protein interaction can be carried out, for example, in high-output diffusion analyses on agar (Young, K. et al., 1998), which allows interactions to be evaluated with respect to a concentration gradient of each inhibitor candidate.

Thus, the present invention relates to a process of identification of a compound inhibiting the interaction of a first protein X with a second protein Y, comprising the following steps:
a) culture cells such as described above,
b) incubate said cells in the presence of the compound to be tested,
c) compare the expression of the reporter gene in the presence and in the absence of said compound, an increase in the expression of the reporter gene being the indication that the compound to be tested is an inhibitor of the interaction of the protein X with the partner protein Y expressed by the cultured cells.

Another application of the interference system according to the present invention is the screening of cDNA banks and of peptide banks in order to identify peptides or protein factors which specifically abrogate a studied protein-protein interaction (Zutshi, R. et al., 1998).

Once a protein-protein interaction pertinent at the level of a disease has been identified and validated, it is interesting to characterize the structure and the regulation of the interaction observed. The identification of mutations at the level of each partner of a pair of proteins in interaction which interrupt the interaction is useful, not only in order to search for the structural components of an interaction, but likewise as a means of generating genetic tools like transdominant negative mutants for the characterization of the function in vivo (Serebriiskii, I. G., et al., 2001). This is particularly important for proteins which have multiple interaction partners. The interference systems according to the present invention can be used in screening by mutagenesis for identification of mutations interrupting an interaction. Banks of interaction partners having undergone mutagenesis must be generated and can be screened in a high-output format concerning re-establishment of cell growth (Gutherie, C., and G. R. Fink (ed.), 2002). By this approach, it is rapidly possible to identify an assembly of deficient mutant proteins at the level of the interaction, especially mutants having dominant negative properties. The assembly of mutants can likewise aid in defining the surfaces of the protein involved in the protein-protein interaction and aid a structural biologist in improving the structure of inhibitor candidates during structure-affinity relationship studies.

The present invention likewise relates to a kit for the setting up of a double-hybrid system such as described above comprising:

An interference DNA construct such as defined above,

A second DNA construct coding for:
A first chimeric protein (Y-AD) formed of a transcription activation domain (AD) fused to a partner protein Y capable of interacting with a partner protein X,

A third DNA construct coding for:
A second chimeric protein (X-DBD) formed of a first DNA-binding domain (DBD) fused to a second domain formed by a protein X capable of interacting with the protein Y, the interaction of the two chimeric proteins X-DBD and Y-AD resulting in the formation of a functional transcription factor activating the interference promoter(s) when the two chimeric proteins are expressed in a host cell.
   The three DNA constructs of the kit described above can be present on the same DNA molecule, or on two or three different DNA molecules. The three constructs can thus be carried by the same DNA vector or are found on two or three different vectors. The two constructs coding for the two chimeric proteins can, for example, be found on the same vector although the third construct is found on a second vector.

The present invention thus provides an assembly of simple genetic systems for the identification of mutations and of molecules which induce the interruption of protein-protein interactions. The present invention is a useful tool for the characterization of protein-protein interactions and likewise for the development of novel medicaments.

The present invention can likewise prove very useful in the case of interaction of a protein Y with two other proteins X and Z and if it is wished to identify molecules inhibiting the interaction between Y and X but not inhibiting the interaction between Y and Z. Cells allowing such a selection carry, for example, the genetic construct present in FIG. 7 and additionally expressing at least three chimeric proteins such as represented in FIG. 7. These cells allow specific inhibitors of an interaction between a protein X and a protein Y to be identified, if the protein Y likewise interacts with another protein Z.

The invention likewise relates to cells containing a DNA construct, so-called interference construct, said construct comprising:

A reporter gene placed under the control of a first inducible promoter,

One or more inducible promoter(s), so-called interference promoter(s), chosen and positioned so that its (their) activation involves transcriptional interference of the first inducible promoter, leading to a detectable decrease in the expression of the reporter gene when the expression of the latter is induced, said cell additionally expressing:
A first chimeric protein (Y-AD) formed of a transcription activation domain (AD) fused to a protein Y, capable of interacting with two partner proteins X and Z,
A second chimeric protein (X-DBD) formed of a first DNA-binding domain (DBD) fused to a second domain formed by a protein X, capable of interacting with the protein Y, the interaction of the two chimeric proteins X-DBD and Y-AD resulting in the formation of a functional transcription factor activating the interference promoter(s),
A third chimeric protein (Z-DBD) formed of a DNA-binding domain (DBD), fused to a second domain formed by a protein Z capable of interacting with the protein Y, the interaction of the two chimeric proteins Z-DBD and Y-AD resulting in the formation of a functional transcription factor activating the expression of the reporter gene.

In a first embodiment, the cells according to the present invention are host cells transformed or transfected by the DNA construct comprising the reporter gene (interference construct) and they are likewise transformed or transfected by three DNA constructs coding for each of the three chimeric proteins. The four constructs can be carried by the same DNA vector or are found on two or three different vectors. All the combinations are conceivable: the four constructs on the same vector, the four constructs on four different vectors, the three constructs coding for the three chimeric proteins on the same vector although the fourth construct (interference construct) is found on a second vector, a construct coding for one of the three chimeric proteins and the interference construct on the same vector although the construct coding for the two other chimeric proteins is found on a second vector etc. . . . . In this first embodiment, the vectors are nonintegrative vectors.

In a second embodiment, the interference construct is integrated into the genome of the cells and the latter are transformed or transfected by three DNA constructs coding for each of the three chimeric proteins. The DNA constructs coding for the chimeric proteins can be carried by the same DNA vector or are found on different vectors, this or these vectors being nonintegrative vectors.

In a third embodiment of the invention, the interference construct as well as the DNA constructs coding for the chimeric proteins are integrated into the genome of the cells. It is likewise possible that only one or two of the three constructs coding for one or two of the three chimeric proteins are integrated into the genome and that the construct(s) may be carried by a nonintegrative vector although the interference construct is integrated into the genome. Finally, in a last embodiment, the interference construct and one or two of the constructs coding for one or two of the three chimeric proteins are carried by nonintegrative vectors although the construct coding for the third chimeric protein is integrated into the genome of the cells.

The reporter gene is such as defined above. The reporter gene is placed under the control of an inducible promoter. Preferably, the inducible promoter regulating the expression of the reporter gene comprises a sequence capable of interacting with a protein having a DNA-binding domain (DBD) (DNA sequence bound by the DBD (UAS)). More particularly, the inducible promoter regulating the expression of the reporter gene comprises a sequence capable of interacting with the DNA-binding domain (DBD) of the chimeric protein (Z-DBD).

The promoter(s), so-called ‘interference promoters’, are likewise inducible promoters. The latter are chosen and positioned so that their activation involves transcriptional interference of the first promoter, leading to a detectable decrease in the expression of the reporter gene when the expression of the latter is induced.

The interference promoters which can be employed are inducible promoters such as defined above. Preferably, the interference promoters which can be employed comprise a sequence capable of interacting with a protein having a DNA-binding domain (DBD) (DNA sequence bound by the DBD (UAS)). More particularly, the inducible interference promoters comprise a sequence capable of interacting with the DNA-binding domain (DBD) of the chimeric protein (X-DBD).

The interference promoter(s) can be positioned as described above. Thus, they can be positioned downstream of the first promoter and of the reporter gene; it is then a question of downstream interference. The interference promoter(s) can likewise be positioned upstream of the first promoter and of the reporter gene; it is then a question of upstream interference.

In the context of downstream interference, the interference promoter can, however, have two possible orientations. A first orientation corresponds to a convergent orientation with respect to the first promoter, this configuration is called downstream interference (or DI). The interference promoter can likewise have a paired orientation with respect to the first promoter, this second configuration has been called nonsense downstream interference (or nDI).

The interference promoter(s) can likewise be positioned upstream of the first promoter and of the reporter gene. The interference promoter then has an orientation identical to that of the first promoter (the two promoters thus have a paired configuration), this configuration has been called upstream interference (or UI).

Another possible configuration is the combination of the two configurations described above (UI and DI system). In this case, at least one (inducible) interference promoter is positioned on both sides of the first inducible promoter and of the reporter gene. It is then a question of upstream-downstream interference because the activation of the interference promoter(s) situated upstream interfere(s) with the activity of the first promoter situated further downstream and the activation of the interference promoter(s) situated downstream interfere(s) with the activity of the first promoter situated further upstream.

The interference promoter(s) situated downstream of the first promoter and of the reporter gene have a convergent orientation with respect to the first promoter (cf. the DI system above). The interference promoter(s) positioned upstream of the first promoter and of the reporter gene have an orientation identical to that of the first promoter (the two promoters thus have a paired configuration) (cf. UI system above).

This configuration has been called upstream-downstream interference (or UDI).

Another possible configuration is the combination of two configurations described above (UI and nDI system). In this case, at least one inducible interference promoter is positioned on both sides of the first inducible promoter and of the reporter gene. It is then a question of upstream-downstream interference because the activation of the interference promoter(s) situated upstream interfere(s) with the activity of the first promoter situated further downstream and the activation of the interference promoter(s) situated downstream interfere(s) with the activity of the first promoter situated further upstream.

The interference promoter(s) situated downstream of the first promoter and of the reporter gene have a paired orientation with respect to the first promoter (cf. the nDI system above). The interference promoter(s) positioned upstream of the first promoter and of the reporter gene have an orientation identical to that of the first promoter (cf. UI system above), the three promoters thus have a paired configuration.

This configuration has been called nonsense upstream-downstream interference (or nUDI).

In the case of a construct of type UI, UDI and nUDI, a short open reading frame followed by one or more (for example 2 or 3) stop codons is preferentially inserted between the interference promoter and the first promoter, as described above.

Preferentially, the interference constructs are bordered at their ends by bidirectional transcription terminators such as described above.

The interference promoter is an inducible promoter which is activated conditionally. In the context of the present invention, it is activated by a transcription factor when this is active. According to the present invention, the transcription factor activating the inducible promoter is formed by an assembly of two chimeric proteins (X-DBD and Y-AD) reconstituting an active transcription factor capable of activating the inducible promoter when an interaction has taken place between the two chimeric proteins X-DBD and Y-AD.

As has been specified above, in this particular embodiment of the present invention, the cells express three chimeric proteins. They thus express:
A first chimeric protein (Y-AD) formed of a transcription activation domain (AD) fused to a partner protein Y, capable of interacting with two partner proteins X and Z,
A second chimeric protein (X-DBD) formed of a first DNA-binding domain (DBD) fused to a second domain formed by a protein X, capable of interacting with the protein Y, the interaction of the two chimeric proteins X-DBD and Y-AD resulting in the formation of a functional transcription factor activating the interference promoter(s),
A third chimeric protein (Z-DBD) expressed is formed of a DNA-binding domain (DBD), fused to a second domain formed by a protein Z capable of interacting with the protein Y, the interaction of the two chimeric proteins Z-DBD and Y-AD resulting in the formation of a functional transcription factor activating the expression of the reporter gene.

The DNA-binding domain (DBD) of the second chimeric protein is chosen as a function of the interference promoter so as to bind the DNA at the level of said promoter. Preferably, the DNA-binding domain (DBD) of the second chimeric protein is chosen in order to interact with a sequence present in said promoter (DNA sequence found by the DBD (UAS) (upstream activating sequence)). The inducible interference promoter(s) comprise a sequence capable of interacting with the DNA-binding domain (DBD) of the chimeric protein (X-DBD).

The transcription activation domain of the first chimeric protein (domain AD of Y-AD) allows the transcription of the interference promoter in the cells to be activated. Thus, the interference promoter(s) (inducible promoters) are induced by the functional transcription factor formed the two chimeric proteins x-DBD and Y-AD interacting between them. This functional transcription factor interacts via its DNA-binding domain (DBD) with a sequence of the interference promoter and is capable of initiating the transcription governed by this promoter via its transcription activation domain (AD).

This forms the very principle of the double-hybrid technique and the person skilled in the art is able to choose multiple promoter/transcription factor pairs allowing the implementation of the present invention, as specified above.

The DNA-binding domain (DBD) of the third chimeric protein (Z-DBD) is chosen as a function of the inducible promoter regulating the expression of the reporter gene, so as to bind the DNA at the level of said promoter. Preferably, the DNA-binding domain (DBD) of the chimeric protein Z-DBD is chosen in order to interact with a sequence present in the promoter regulating the expression of the reporter gene (DNA sequence bound by the DBD (UAS) (upstream activating sequence)).

The inducible promoter regulating the expression of the reporter gene and the interference promoter(s) are different promoters. The inducible promoter regulating the expression of the reporter gene is especially chosen so as not to bind the chimeric protein X-DBD (this promoter thus does not contain any DNA sequence bound by the DBD (UAS) (upstream activating sequence) of X-DBD)). In addition, the inducible interference promoter(s) are chosen so that they do not bind the chimeric protein Z-DBD (this promoter thus does not contain any DNA sequence bound by the DBD (UAS) (upstream activating sequence) of Z-DBD)).

The transcription activation domain of the first chimeric protein (domain AD of Y-AD) allows the transcription of the promoter regulating the expression of the reporter gene in the cell to be activated. Thus, the promoter regulating the expression of the reporter gene is induced by the functional transcription factor formed the two chimeric proteins Z-DBD and Y-AD interacting between them. This functional transcription factor interacts via its DNA-binding domain (DBD) with a sequence of the promoter regulating the expression of the reporter gene and is capable of initiating the transcription governed by this promoter via its transcription activation domain (AD).

This forms the very principle of the double-hybrid technique and the person skilled in the art is able to choose multiple promoter/transcription factor pairs allowing the implementation of the present invention, as described above.

Thus, in the case of interaction between the proteins Y and X, the interference promoter is activated, which leads to interference in the transcription of the reporter gene. If the interaction between Y and X is perturbed (for example by the action of a chemical inhibitor inhibiting the interaction between Y and X), the interference promoter will be less activated or not activated, thus leading to an increased expression of the reporter gene.

The partner proteins X, Y and Z fused to the DNA-binding domain (DBD) and to the of a transcription activation domain (AD) can be defined as any group of proteins for which an interaction exists between Y and X and between Z and Y and which, when Y is fused to AD and Z and X are fused to different DBDs, interact in the cell (even if that is not the case in their organisms of origin) in order to result in the formation of two functional transcription factors (X-DBD/Y-AD and Z-DBD/Y-AD respectively) activating the inducible promoters such as described above. They can be complete proteins or fragments of the latter. Thus, the chimeric proteins generating a double-hybrid signal can be used in the context of the present invention. That is to say the chimeric proteins whose two partner proteins (or fragments of the partner proteins) interact between them with a sufficient affinity in the cell used, in order to form a functional transcription factor activating the inducible promoter(s), said transcription factor binding the DNA via the binding domain to of the first chimeric protein and activating the promoter(s) by the transcription activation domain (AD) of the second chimeric protein.

When the interference DNA construct is integrated into the genome of the host cell, this construct is advantageously targeted towards a locus free of potentially perturbatory genomic transcription activities, as described above.

The cells which can be used in the context of this embodiment of the invention (three chimeric proteins) are those described above.

As has been specified above, the double-hybrid system according to the present invention can have numerous applications. Thus, it is particularly appropriate for the screening of molecules having an activity of inhibition of a protein-protein interaction. In the context of the embodiment of the invention with three chimeric proteins, the cells described above are particularly interesting as far as the identification of molecules inhibiting the interaction between the proteins Y and X but not inhibiting the interaction between Y and Z is concerned. Thus, in these cells, the molecules dissociating the protein-protein interaction between Y and X cause the expression of the reporter gene which allows their identification.

The present invention thus likewise relates to a process for identification of a compound inhibiting the interaction of a first protein X with a second protein Y, but not inhibiting or inhibiting less the interaction between the protein Y and a third protein Z, comprising the following steps:
a) culture cells such as described above (three chimeric proteins embodiment),
b) incubate said cells in the presence of the compound to be tested,
c) compare the expression of the reporter gene in the presence and in the absence of said compound, an increase in the expression of the reporter gene being the indication that the compound to be tested is an inhibitor of the interaction of the protein X with the partner proteins Y, but that this product does not inhibit or inhibits less the interaction between the protein Y and the protein Z.

Another application of the three chimeric proteins interference system is the screening of cDNA banks and of banks of peptides in order to identify peptides or protein factors which specifically abrogate the interaction between the protein X and the protein Y studied (Zutshi, R. et al., 1998), but which do not affect the interaction between the protein X and the protein Z.

Another application is the identification of mutations in the protein Y which interrupt the interaction between the protein Y and the protein X, but which do not affect the interaction between the protein Y and the protein Z. This is useful not only in order to search the structural components of a particular interaction, but likewise as a means of generating genetic tools like transdominant negative mutants for the characterization of the in vivo function (Serebriiskii, I. G., et al., 2001) (see above).

The present invention likewise relates to a kit for the setting up of a double-hybrid system such as described above comprising:

A DNA construct such as defined above,

A second DNA construct coding for:
A first chimeric protein (Y-AD) formed of a transcription activation domain (AD) fused to a partner protein Y, capable of interacting with two partner proteins X and Z,

A third DNA construct coding for:
A second chimeric protein (X-DBD) formed of a first DNA-binding domain (DBD) fused to a second domain formed by a protein X, capable of interacting with the protein Y, the interaction of the two chimeric proteins X-DBD and Y-AD leading to the formation of a functional transcription factor activating the interference promoter(s),

A fourth DNA construct coding for:
A third chimeric protein (Z-DBD) formed of a DNA-binding domain (DBD), fused to a second domain formed by a protein Z capable of interacting with the protein Y, the interaction of the two chimeric proteins Z-DBD and Y-AD leading to the formation of a functional transcription factor activating the expression of the reporter gene.
   The four DNA constructs of the kit described above can be present in the same DNA molecule, or on two or three or four different DNA molecules. The four constructs can thus be carried by the same DNA vector or are found on two, three or four different vectors. The three constructs coding for the three chimeric proteins can, for example, be found on the same vector although the fourth construct is found on a second vector.

The present invention is a useful tool for the characterization of protein-protein interactions and but likewise for the development of novel medicaments.

### KEY TO THE FIGURES

FIG. 1: rY2H Systems Based on Transcription Interference.

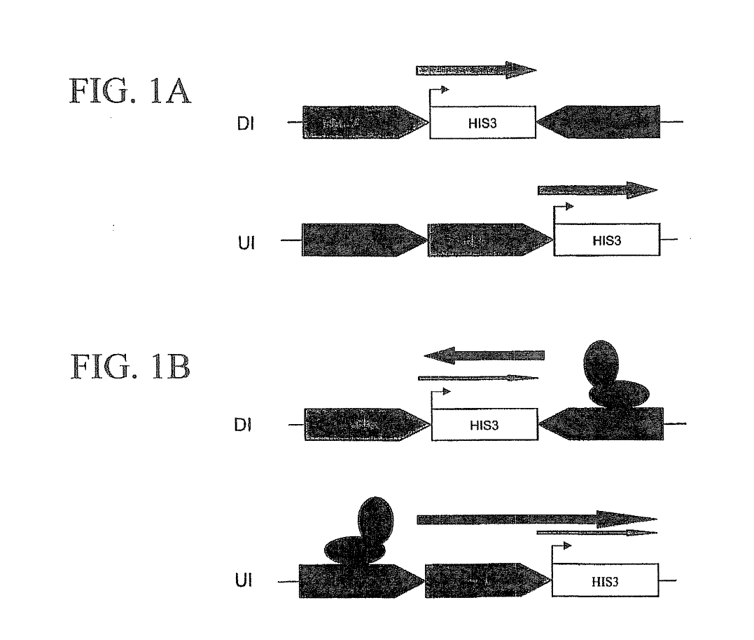

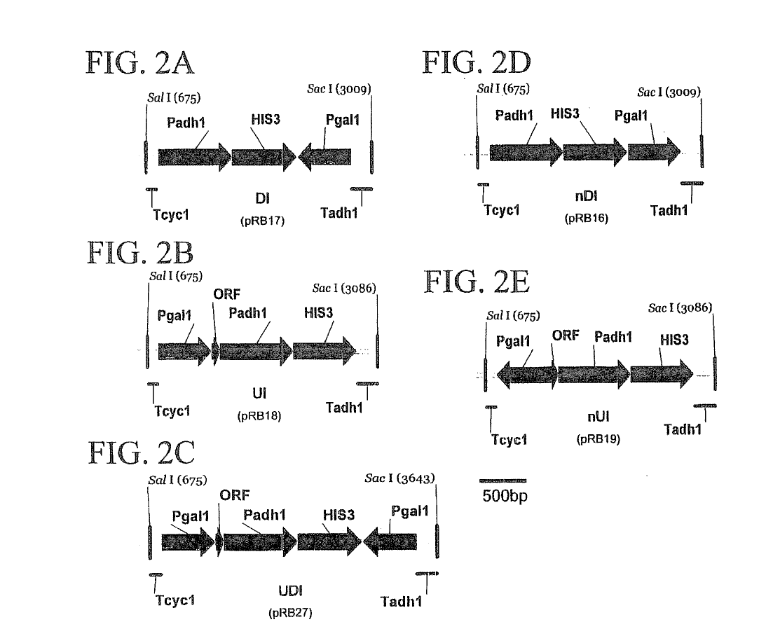

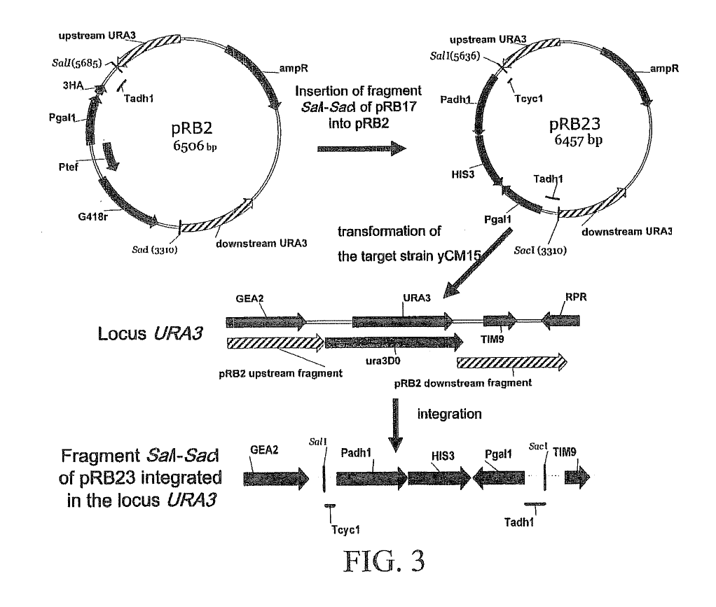

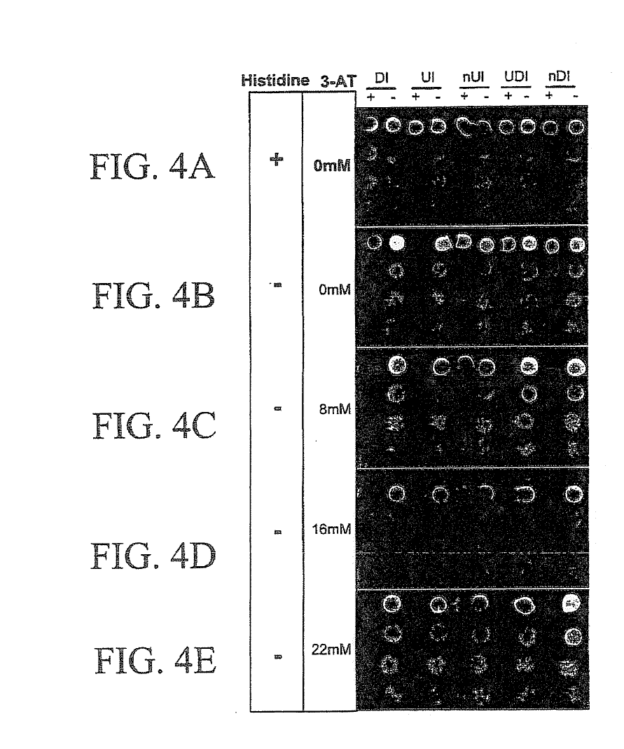

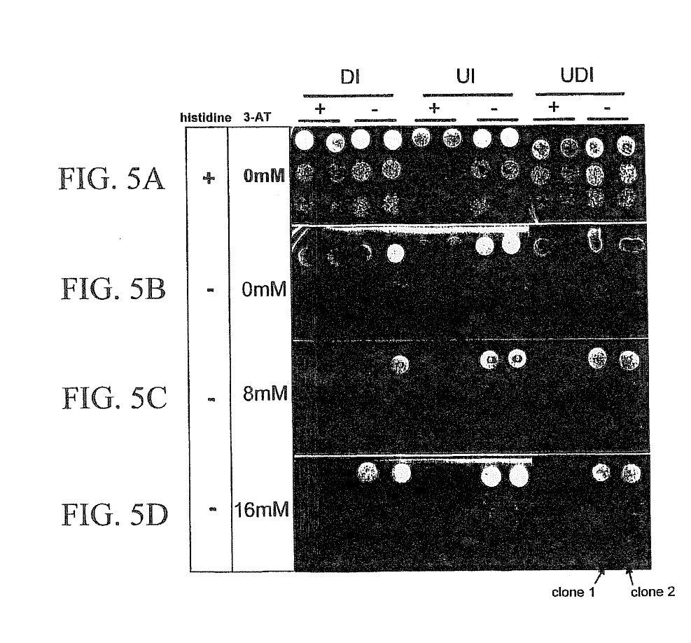

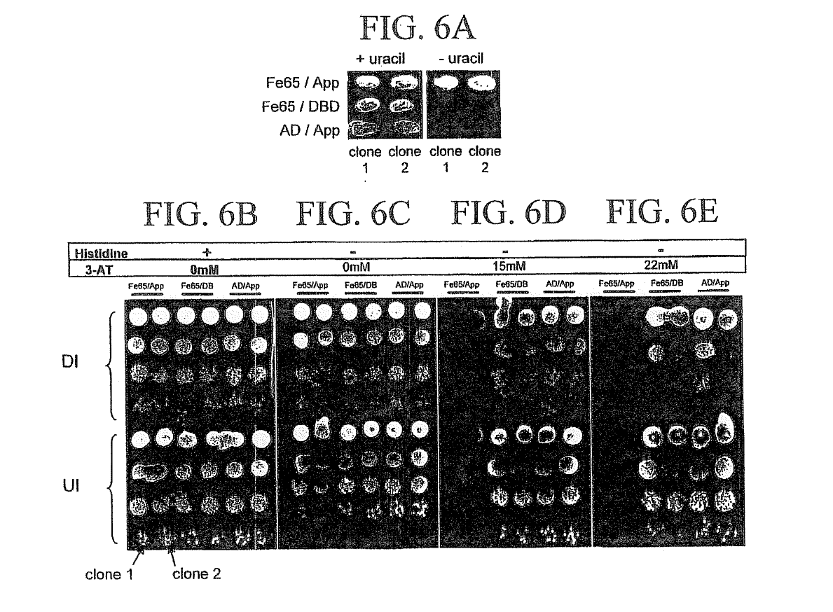

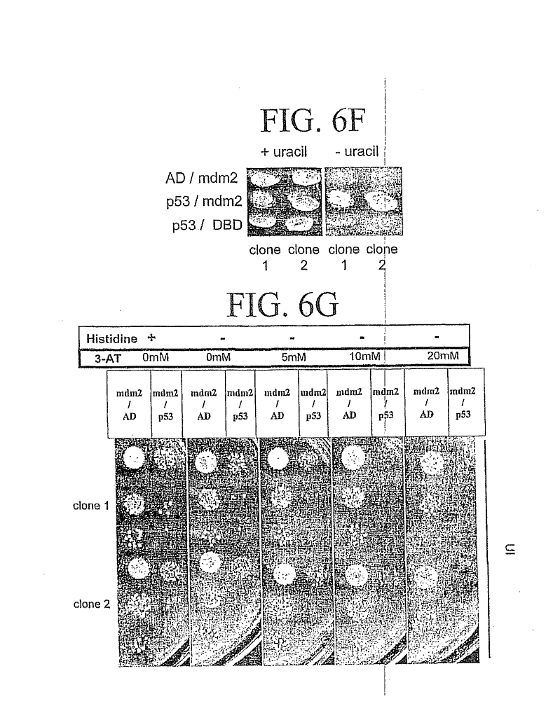

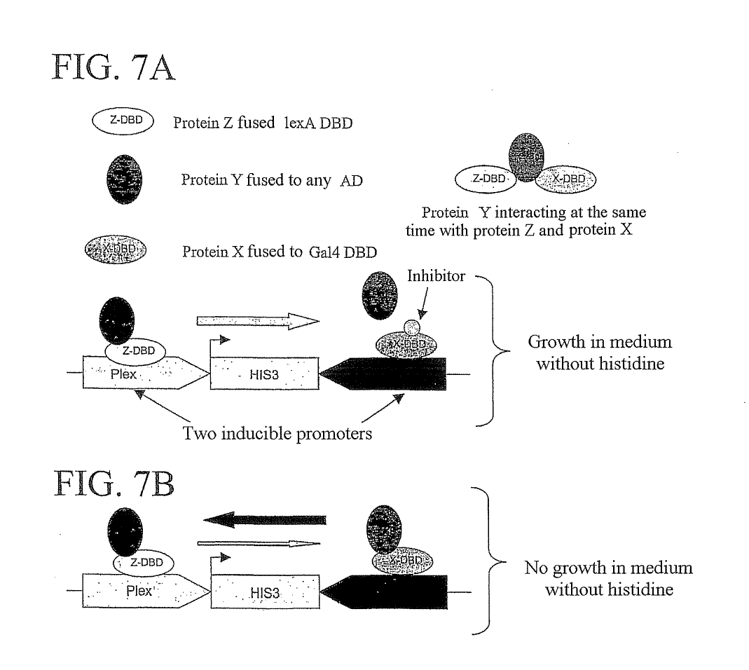

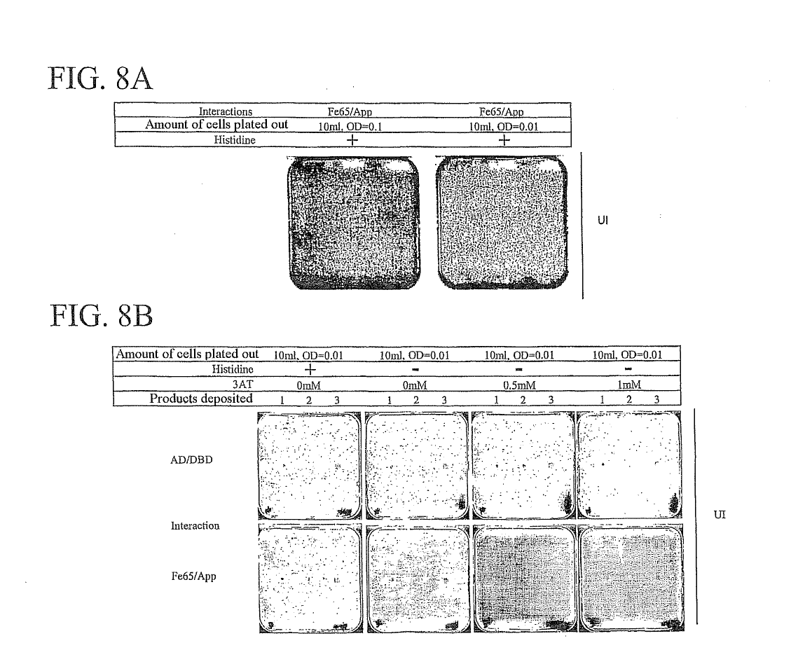

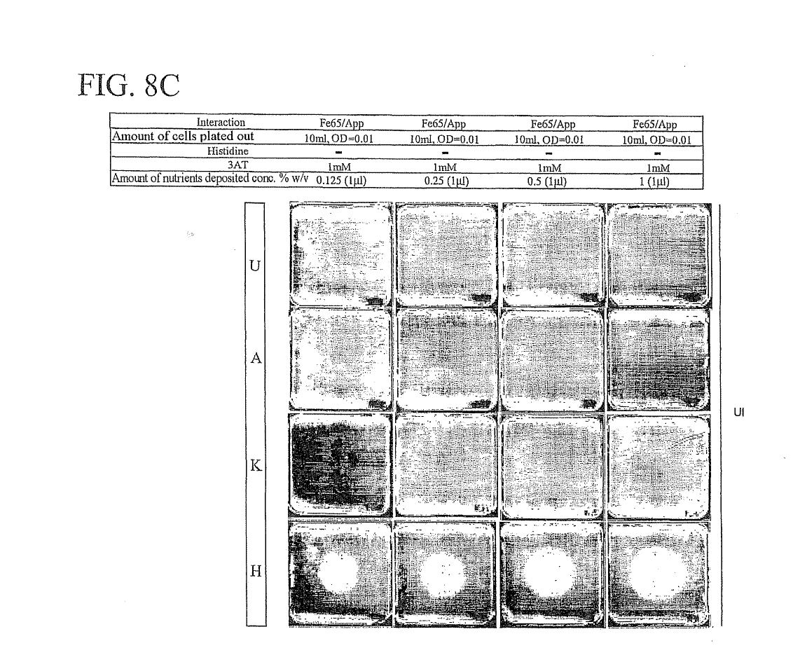

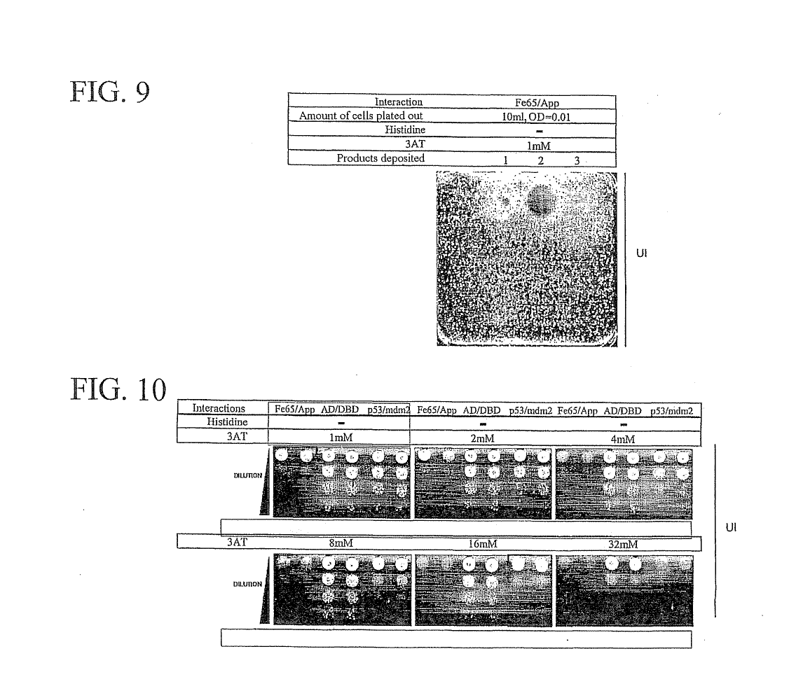

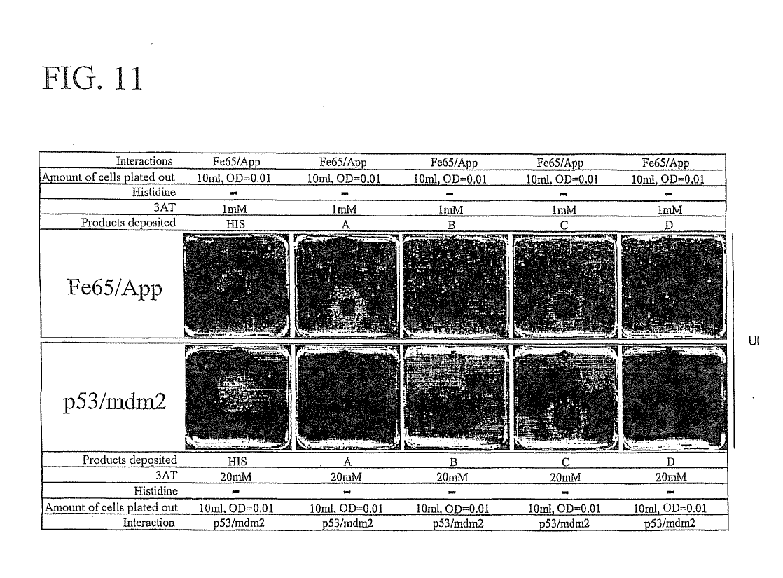

Schematic presentation illustrating the application of transcription interference for the detection of reverse Y2H interactions. In the absence of a Y2H interaction, the reporter gene HIS3 is expressed normally and the cells can grow on a medium without histidine (FIG. 1A). The presence of a Y2H interaction reduces the level of transcription of HIS3, which causes a reduction in cell growth of the auxotrophic reporter strain for histidine (FIG. 1B). The ORF of HIS3 is symbolized by the rectangle of light color, the small black arrow marking the position of the start codon. The grey arrow above, of the ORF of HIS3 symbolizes the transcription initiated at the level of Padh1 whereas the black arrow represents the transcription of Pgal1. The thickness of the arrows symbolizes the transcription activity of the promoters. The arrows point in the direction of the transcription. Proteins X and Y in interaction used for GAL4-DBD (X-DBD) and GAL4-AD (Y-AD) are shown in ovoid form. Padh1: ADH1 promoter; Pgal1: GAL1 promoter; HIS3: ORF of HIS3; DI: downstream interference; UI: upstream interference. Figure not shown to scale.

FIG. 2.

Transcription Interference Constructs.

Five different constructs have been employed for the setting up of the rY2H systems based on transcription interference; the five constructs are shown to scale (FIG. 2A to 2E). The restriction sites SalI and SacI and their positions on the corresponding pBluescript vectors are indicated. The names of the plasmids exemplified (see example 4 and table 2) are indicated. Abbreviations: Padh1: ADH1 promoter; Pgal1: GALL promoter; HIS3: ORF of HIS3; Tadh1: ADH1 terminator; Tcyc1: CYC1 terminator; ORF: open reading frame followed by two stop codons; DI: downstream interference (2A); UI: upstream interference (2B); UDI: upstream-downstream interference (2C); nDI: nonsense downstream interference (2D); nUI: nonsense upstream interference (2E).

FIG. 3.

Genomic Integration of Interference Constructs.

Example illustrating the genomic integration of transcription interference constructs. The integration vector pRB23 was constructed by inserting the fragment SalI-SacI of pRB17 into pRB2. pRB23 is then used in order to transform the target strain yCM15 where it is integrated at the level of the locus URA3 by homologous recombination. The homologous vector sequences at the locus of URA3 (upstream/downstream of URA3) are shown hatched. The ORFs adjacent to the gene of URA3 are shown (GEA2, TIM9 and RPR) and the model deletion ura3D0 is indicated (Brachmann, C. B., et al., 1998). Abbreviations: Padh1: ADH1 promoter; Pgal1: GALL promoter; HIS3: ORF of HIS3; Tadh1: ADH1 terminator; Tcyc1: CYC1 terminator; 3HA: coded tag sequences on pRB2; Ptef: TEF promoter; G418r: ORF coding for resistance to G418; ampR: ampicillin resistance gene.

FIG. 4.

Functional Evaluation of Integrated Interference Systems.

The strains carrying integrated copies of interference systems (yRB48 (DI), yRB49 (UI), yRB50 (nUI), yRB83 (UDI) and yRB82 (nDI)) are transformed with the series of plasmids pCL1/pFL39 (+) and pTAL20/pDBT (−), respectively, and the cell growth of the transformants is studied on complete medium (FIG. 4A) or medium depleted in histidine (FIG. 4B to E) by a drop evaluation method. Dilution series of a factor 10 are prepared for each transformant. A competitive inhibitor of the protein HIS3p (3-amino-1,2,4-triazole (3-AT)) is optionally added to the culture medium at growing concentrations as indicated (0 mM to 22 mM) (FIG. 4B to E). Abbreviations: DI: downstream interference strain; UI: upstream interference strain; nDI: nonsense downstream interference strain; nUI: nonsense upstream interference strain; UDI: upstream-downstream interference strain.

FIG. 5.

Functional Evaluation of Interference Systems Based on Plasmids (Not Integrated at the Genomic Level):

The strain yCM15 is cotransformed independently with versions based on plasmids of interference systems (pRB20 (DI), pRB21 (UI) or pRB28 (UDI)) and the series of plasmids pCL1/pFL39 (+) and pTAL20/pDBT (−), respectively. The cell growth of each of these triple transformants is studied on agar plates with (FIG. 5A) and without (FIG. 5B to D) histidine and, for the plates without histidine, without or with growing concentrations of 3-AT (0 mM to 16 mM) (FIG. 5B to D). Dilution series of a factor 10 are prepared and two independent clones are evaluated for each transformed strain. Abbreviations: DI: downstream interference plasmid; UI: upstream interference plasmid; UDI: upstream-downstream interference plasmid.

FIG. 6.

Transcription Interference Induced by Y2H Interactions.

FIGS. 6A and 6F:

The protein-protein interactions previously identified between the proteins Fe65 and App and p53 and mdm2 sufficiently induce a transcription interference construct in order to inhibit the cell growth of yeast.

The activity of the constructs is first verified (FIGS. 6A and 6F). In order to do that, the reporter strain Y187 is transformed by the following plasmid pairs: pRB34+pRB35 (Fe65/App), pRB34+pDBT (Fe65/DBD) and pTAL20+pRB35 (AD/App) (FIG. 6A) and pDBT-mdm2+pVP16-p53Nt (p53/mdm2), pVP16-p53Nt+PDBT (p53/DBD) and pTAL20+pDBT-mdm2 (AD/mdm2) (FIG. 6F). Two clones of each of these transformants are cultured on minimum medium with and without uracil ((FIGS. 6A and 6F).

The strains yRB48 (system DI) and yRB49 (system UI) are then each cotransformed by the plasmid pair pRB34 (Fe65) and pRB35 (App) allowing the expression of the two hybrid proteins AD-Fe65 and DBD-AppCt. The strains yRB48 (system DI) and yRB49 (system UI) are likewise each cotransformed by the plasmid pairs pRB34+pDBT (Fe65/DBD) and pTAL20+pRB35 (AD/App) as controls.

The cell growth of two clones of each of the double transformants is studied on agar plates with and without histidine and, for the plates without histidine, without or with growing concentrations of 3-AT (FIG. 6B to 6E). The strain yRB49 (system UI) is likewise cotransformed by the plasmid pair pDBT-mdm2+pVP16-p53Nt (p53/mdm2) allowing the expression of the two hybrid proteins DBD-mdm2 and VP16-p53 (1-55). The strain yRB49 (system UI) is likewise cotransformed by the plasmid pair pDBT-mdm2+pTAL20 (AD/mdm2) as controls. Two clones cotransformed by the two plasmids are selected for each of these two plasmid pairs.

The cell growth of each of the two clones of these double transformants is studied on agar plates with and without histidine and for the plates without histidine, without or with growing concentrations of 3-AT, as indicated on the figure.

FIG. 7

rY2H Systems Based on Transcription Interference According to the Present Invention Allowing the Identification of Molecules Inhibiting the Interaction Between Two Proteins Y and X but not Inhibiting the Interaction Between the Protein Y and a Third Protein Z

Schematic presentation illustrating the application of transcription interference for the detection of reverse Y2H interactions allowing the identification of molecules inhibiting the interaction between two proteins Y and X but not inhibiting the interaction between the protein Y and a third protein Z. In the absence of an interaction Y2H between the protein X and Y, the reporter gene HIS3 is expressed normally and the cells can grow on a medium without histidine (FIG. 7A). This is the case when an inhibitor of the interaction of the protein X with the partner protein Y is present. The presence of a Y2H interactions between X-DBD and Y-AD reduces the level of transcription of HIS3, which causes a reduction in the cell growth of the auxotrophic reporter strain for histidine (FIG. 7B). The ORF of HIS3 is symbolized by the rectangle of light color, the small black arrow marking the position of the start codon. The gray arrow above the ORF of HIS3 symbolizes the transcription initiated at the level of Plex whereas the black arrow shows the transcription of Pgal1. The thickness of the arrows symbolizes the transcription activity of the promoters. The arrows point in the direction of transcription. Proteins in interaction X/Y and Z/Y used are shown in ovoid form. Plex: LexA promoter; Pgal1: GALL promoter; HIS3: ORF of HIS3; Figure not shown to scale.

FIG. 8:

Determination of the Experimental Conditions for Screening for Inhibitors of the Fe65/App Interaction

FIG. 8A:

Determination of the Amount of Cells:

The strain containing the UI transcriptional interference system (yRB49) was cotransformed with the plasmids pRB34 and pRB35 (Fe65/App interaction). A culture of the transformed strain was diluted to OD=0.1 and 0.01, and 10 ml of each dilution were plated out onto medium supplemented with histidine (+HIS). The dishes were incubated at 30° C. for 48 hours.

FIG. 8B:

Determination of the Concentration of 3-AT:

The strain containing the UI transcriptional interference system (yRB49) cotransformed with the plasmids pRB34 and pRB35 (Fe65/App interaction) or with the control plasmids pTAL20 and pDBT (AD/DBD) was plated out onto medium with histidine and without 3-AT, without histidine and without 3-AT, and with histidine and with 0.5 mM or 1 mM of 3-AT. 1 μl of 100% DMSO, 1 μl of a solution of a molecule x at 10 mM in DMSO, and 1 μl of a solution of a molecule y at 10 mM in DMSO were then deposited onto each dish. The dishes were incubated at 30° C. for 48 hours.

FIG. 8C:

Restoration of Growth by Histidine:

The strain containing the UI transcriptional interference system (yRB49) and cotransformed with the plasmids pRB34 and pRB35 (Fe65/App interaction) was plated out onto media without histidine and containing 1 mM of 3-AT. 1 μl of uracil (U), of adenine (A), of lysine (K) or of histidine (H), at 0.125%, 0.25%, 0.5% or 1%, was deposited at the center of each dish. The dishes were incubated at 30° C. for 48 hours.

FIG. 9:

Detection of Inhibitors of the Fe65/App Interaction by Restoration of Growth

The strain containing the UI transcriptional interference system (yRB49) and cotransformed with the plasmids pRB34 and pRB35 (Fe65/App interaction) was plated out onto medium without histidine and containing 1 mM of 3-AT. 1 μl of a 10 mM solution of a molecule 1, which is a putative inhibitor of the Fe65/App interaction, 1 ul of a 10 mM solution of a molecule 2, which is a putative inhibitor of the Fe65/App interaction, and 1 μl of a 10 mM solution of a molecule 3, which is inactive on the Fe65/App interaction were deposited onto this dish. The dishes were incubated at 30° C. for 48 hours.

FIG. 10

Comparison of the Fe65/App and p53/MDM2 Interactions for the Activation of the Interference System

The strain containing the UI transcriptional interference system (yRB49) was cotransformed with the pairs of plasmids pRB34 and pRB35 (Fe65/App interaction), pDBT-mdm2 and pVP16-p53Nt (mdm2/p53 interaction) or pTAL20 and PDBT (control). For each transformation, two independent clones were cultured. After incubation at 30° C. overnight, all the cultures were brought to the same OD. Ten-fold serial dilutions of each culture were carried out and a drop of each dilution was deposited onto medium without histidine and supplemented with various concentrations of 3-AT (from 1 to 32 mM). The dishes were incubated at 30° C. for 48 hours.

FIG. 11:

Confirmation of the Specificity of the Inhibitors of the Fe65/App Interaction

The strain containing the UI transcriptional interference system (yRB49) was cotransformed with the pairs of plasmids pRB34 and pRB35 (Fe65/App interaction) or pDBT-mdm2 and pVP16-p53Nt (mdm2/p53 interaction). The strain containing the plasmids pRB34 and pRB35 (Fe65/App interaction) was plated out onto 5 dishes of medium without histidine supplemented with 1 mM of 3-AT (top row); the strain containing the plasmids pDBT-mdm2 and pVP16-p53Nt (mdm2/p53 interaction) was similarly plated out onto 5 dishes of medium without histidine supplemented with 20 mM of 3-AT (bottom row).

A control dish was prepared for each interaction by depositing 1 μl of histidine at 0.125% w/v at the center of this dish. A different molecule, A, B, C or D, was deposited onto each of the other 4 dishes, according to the following scheme: in the upper left corner, 1 μl of the product at a concentration of 0.1 mM is deposited; in the upper right corner, 1 μl of the same product at a concentration of 1 mM is deposited; at the center, 1 μl of this product at 10 mM is deposited.

The dishes were incubated at 30° C. for 48 hours.

The present invention is illustrated with the aid of the examples which follow, which must be considered as illustrative and non-limiting.

The techniques of molecular biology used in the examples which follow correspond to standard protocols of molecular biology and are described by (Sambrook, J. et al., 1989). The DNA sequences were amplified by using a high fidelity PCR system (Expand High Fidelity PCR System, Roche Diagnostics, Penzberg, Germany).

### EXAMPLE 1

### Construction of the Plasmid pRBV

A fragment of 761 bp corresponding to the complete constitutive promoter of the gene ADH1 is generated by PCR by using the following oligonucleotides as primers: RB43 5′-AAAATCTAGAGGCGCCATATCCTTTTGTTGTTTCCGG-3′ (SEQ ID No. 1) and RB44 5′-AAAAACGCGTGGCGCCCATCTTTCAGGAGGCT TGC-3′ (SEQ ID No. 2) (the restriction sites XbaI and MluI are underlined) and as genomic DNA matrix of the strain FL100 of S. cerevisiae (accessible especially from the American Type Culture Collection (ATCC) (Manassas, Va., USA), under the number 28383).

Two phosphorylated single-strand oligonucleotides: RB75 of sequence 5′-CGCGTGGGGGGTTAATTAAAAAAAAGC-3′ (SEQ ID No. 3) and RB76 of sequence 5′-GGCCGCTTTTTTTTAATTAACCCCCCA-3′ (SEQ ID No. 4) are hybridized in order to obtain a double-stranded linkage arm (linker) having compatible cohesive extremities MluI and NotI, respectively at the ends. In order to do that, the two single-strand oligonucleotides are mixed in water at equimolar concentrations. The mixture is heated to 90° C. for 5 min in a heating block (heat block) and then the block is allowed to cool to ambient temperature (approximately 2 hours).

In order to construct the vector pRB3, the fragment of 761 bp obtained by PCR (fragment Padh1) is first of all digested by the restriction enzymes XbaI and MluI. The fragment digested in this way is then inserted conjointly with the linker into the vector pBluescript KS+(marketed by Stratagene (LaJolla, Calif., USA)) previously digested by XbaI-NotI. The plasmid thus obtained is called pRB3.

A fragment of 660 bp corresponding to the open reading frame of the gene HIS3 is then amplified by PCR by using as matrix genomic DNA of the strain FL100 of S. cerevisiae and as primers the following oligonucleotides: RB45 5′-AAAA ACGCGTACAGAGCAGAAAGCCCTAG-3′ (SEQ ID No. 5) and RB46 5′-AAAAAAGCGGCCGCGGCGCGCCTTAATTAACTACATAAGAACACCTTTGGTG-3′ (SEQ ID No. 6) (the sites Mlul, NotI and AscI are underlined). This fragment was digested by the restriction enzymes MluI and NotI and cloned into the vector pRB3 previously linearized by the restriction enzymes MluI and NotI. The vector thus obtained was called pRB4.

A PCR fragment of 203 bp coding for the terminator of the gene ADH1 (Tadh1) is then amplified by PCR by using as matrix genomic DNA of the strain FL100 of S. cerevisiae and the following oligonucleotides as primers: RB51 5′-AAAAGGCGCGCCTAATTCCGGGCGAATTTCT-3′ (SEQ ID No. 7) and RB52 5′-AAAAGAGCTCTGCATGCCGGTAGAGGTG-3′ (SEQ ID No. 8) (the sites AscI and SacI are underlined). The fragment obtained is digested by the restriction enzymes AscI and SacI and it is inserted into the vector pRB4 previously digested by the enzymes AscI and SacI. The plasmid thus obtained is called pRB5.

A fragment of 88 bp coding for the terminator of the gene CYC1 (Tcyc1) is obtained by hybridization of two phosphorylated single-strand oligonucleotides RB98 of sequence: 5′-CGATCGCGTTTGTACAGAAAAAAAGAAAAATTTGAAATAT AAATAACGTTCTTAATACTAACATAACTATT AAAAAAATAAATAGGGACCG-3′ (SEQ ID No. 9) and RB99 of sequence: 5′-AATTCGGTCCCTATTTATTTTTTT TAATAGTTATGTTAGTATTAAGAACGTTATTTATATTTCAAATTTTTCTTTTTTTTCT GTACAAACGCGAT-3′ (SEQ ID No. 10). In order to do that, the two single-strand oligonucleotides are mixed at equimolar concentrations in water. The mixture is heated to 90° C. for 5 min in a heating block (heat block) and then the block is allowed to cool to room temperature (approximately 2 hours). The double-stranded fragment thus obtained has compatible cohesive extremities EcoRI and ClaI respectively at its ends. This allows the insertion of this double-stranded fragment (corresponding to the terminator Tcyc1) into the vector pRB5 described above previously digested by the enzymes EcoRI and ClaI. The vector thus obtained is called pRBV.

The sequence of this construct is verified by sequencing with the aid of the ‘Big Dye Terminator’ kit (Perkin-Elmer, Wellesley, Mass., USA).

### EXAMPLE 2

### Construction of Transcriptional Interference Systems

2.1 Construction of the Downstream Interference Vector pRB17 and of the Nonsense Downstream Interference Vector pRB16:

The downstream interference vector pRB17 (also called DI) and the nonsense downstream interference vector pRB16 (also called nDI) are obtained by inserting a fragment of PCR of 549 bp corresponding to the promoter Pgal1 into the vector pRBV (cf. example 1).

This PCR fragment is obtained by using as matrix genomic DNA of the strain FL100 of S. cerevisiae and the following oligonucleotides as primers: RB49 5′-AAAAGGCGCGCCGTAAAGAGCCCCATTATCTTAG-3′ (SEQ ID No. 11) and RB50 5′-AAAAGGCGCGCCTTTGAGATCCGGGTTTTTTCT-3′ (SEQ ID No. 12) (the restriction sites AscI are underlined). This PCR fragment is then digested by the enzyme AscI and inserted into the vector pRBV rendered linear with the enzyme AscI. The fragment can be inserted in both directions which allows the two constructs to be obtained: the downstream interference vector pRB17 (also called DI and in which Padh1 and Pgal1 are orientated in opposite directions) (cf. FIG. 2A) and the nonsense downstream interference vector pRB16 (also called nDI and in which Padh1 and Pgal1 are in the same orientation) (cf. FIG. 2D).

The sequence of these constructs is verified by sequencing with the aid of the ‘Big Dye Terminator’ kit (Perkin-Elmer, Wellesley, Mass., USA).

The unidirectional promoter Pgal1 used comes from the divergent bidirectional promoter GAL1-10 (described by Johnston, M., and R. W. Davis., 1984), and contains four consensus sites ‘GAL4p-responsive DNA enhancer elements’ as well as the base promoter of the gene GAL1, but does not have the TATA box of the promoter of the gene GAL10. The promoter GAL1 is regulated by the transcription factor GAL4p for which a double-hybrid pair (Y2H) is available (separate AD and DBD domains). This promoter is well characterized and has been used with success in applications requiring the inducible expression of a promoter, like for the purification of proteins, the study of the function of an essential gene etc. . . . .

2.2 Construction of the Upstream Interference Vector pRB18 and of the Nonsense Upstream Interference Vector pRB19:

In order to construct the upstream interference vector pRB18 (also called UI) and the nonsense upstream interference vector pRB19 (also called nUI), a short open reading frame coding for the amino acids of the carboxy end of the protein GST (C-ter GST) is generated by hybridization of two single-strand phosphorylated oligonucleotides: RB81 of sequence 5′-CCGGGGCGGCCGCAACGTTTGGTGGTGGCGACCATCCTCCAA AATCGGATCTGGTTCCGCGTTGAGTAGCTGAATAAGTGAATAGGCGGCCGCT-3′ (SEQ ID No. 13) and RB82 of sequence 5′-CTAGAGCGGCCGCCTATTCACT TATTCAGCTACTCAACGCGGAACCAGATCCGATTTTGGAGGATGGTCGCCACCACCAA ACGTTGCGGCCGCC-3′ (SEQ ID No. 14) (the site NolI is underlined).

In order to do that, the two single-strand oligonucleotides are mixed at equimolar concentrations in water. The mixture is heated at 90° C. for 5 min in a heating block (heat block) and then the block is allowed to cool to ambient temperature (approximately 2 hours). The double-stranded fragment thus obtained has at the ends compatible cohesive extremities XmaI and XbaI, respectively.

In parallel, a fragment of 552 bp corresponding to the promoter of the gene GAL1 (it is the same promoter as that used for the construct of the downstream interference vector pRB17, however the fragment is 3 bp longer than that used for the downstream construct since it contains a start codon ATG in order to initiate the translation of the short open reading frame) was then amplified by PCR. This fragment is obtained by using as matrix the genomic DNA of the strain FL100 of S. cerevisiae and the following oligonucleotides as primers: RB55 5′-AAAACCCGGGGTAAAGA GCCCCATTATCTTAG-3′ (SEQ ID No. 15) and RB56 5′-AAAACCCGGGCATTTT GAGATCCGGGTTTTTTC-3′ (SEQ ID No. 16) (the sites XmaI are underlined). This fragment is then digested with the enzyme XmaI.

A three fragment ligation is then carried out (the two fragments obtained above+the vector pRBV). This ligation consists in the insertion of the fragment Pgal1 digested with the enzyme XmaI and of the short open reading frame C-ter GST (compatible cohesive extremities XmaI and XbaI) into the vector pRBV (previously digested by XmaI-XbaI). The fragment Pgal1 can be inserted in both directions. Consequently, two constructs are obtained: the upstream interference vector pRB18 (also called UI and in which Padh1 and Pgal1 are in the same orientation) (cf. FIG. 2B) and the nonsense upstream interference vector pRB19 (also called nUI and in which Padh1 and Pgal1 are orientated in opposite directions) (cf. FIG. 2E).

The sequence of these constructs is verified by sequencing with the aid of the ‘Big Dye Terminator’ kit (Perkin-Elmer, Wellesley, Mass., USA).

2.3 Construction of the Upstream-Downstream Interference Vector pRB27:

The upstream-downstream interference plasmid pRB27 (also called UDI) is constructed by inserting the same fragment of PCR of 549 bp corresponding to the promoter Pgal1 as that used for the construction of pRB16 and pRB17 (cf. example 2: this PCR fragment is obtained by using as matrix the genomic DNA of the strain FL100 of S. cerevisiae and the oligonucleotides RB49 and RB50 as primers) into the vector pRB18. In order to do that, the fragment of PCR is digested by the enzyme AscI and inserted into the vector pRB18 rendered linear by digestion with the enzyme AscI. It being possible to insert the fragment in both directions, a plasmid with the insert in the desired orientation (in which the two promoters Pgal1 are orientated in opposite directions) is more particularly selected (by restriction profile) (cf. FIG. 2C).

The vector thus obtained is called pRB27. The sequence of this construct is verified by sequencing with the aid of the ‘Big Dye Terminator’ kit (Perkin-Elmer, Wellesley, Mass., USA).

2.4 Principle of the Construction of Transcriptional Interference Systems:

The applicant has conceived two types of rY2H (reverse double-hybrid) systems which are both based on gene silencing by transcription interference. In the first configuration, Pgal1 is situated downstream of the gene HIS3, and Padh1 and Pgal1 have a convergent orientation. This configuration is called downstream interference (DI) because the downstream activation of Pgal1 interferes with the transcription of the gene HIS3. In the second configuration, Pgal1 is situated upstream of Padh1 and the two promoters have a paired orientation. Consequently, this configuration is called upstream interference (UI) because the upstream activation of Pgal1 interferes with the activity of the promoter Padh1.

On the basis of the configurations UI and DI, five transcription interference constructs rY2H have been developed (cf. FIG. 2A to 2E).

An activation of Pgal1 in the system UI will lead to a reduction in the activity of Padh1 owing to transcription interference. The activity of Pgal1 could, however, even allow a sense transcript of the gene HIS3 to be produced. In order to prevent the translation of this sense transcript to functional IGP dehydratase, a short open reading frame of 22 amino acids followed by two stop codons was inserted between Pgal1 and Padh1. Consequently, transcription interference in a system UI produces a bicistronic messenger starting from which only the first short open reading frame is translated.
The third construct is a combination of the systems UI and DI. In this system, called an upstream-downstream interference system (UDI), Pgal1 is inserted upstream and paired with respect to Padh1-HIS3, and an additional copy of Pgal1 is placed downstream of the open reading frame of HIS3 and this in a convergent orientation with respect to Padh1 (cf. FIG. 2C).
In order to control the specificity of the transcription interference induced by Pgal1, a nonsense upstream (nUI) and nonsense downstream (nDI) interference system were constructed. In these two control constructs, the orientation of Pgal1 is reversed with respect to the constructs UI and DI, respectively. No transcriptional interference should be observed during the use of these control constructs, even in the presence of a valid Y2H interaction.

The five constructs described above (the five inserts of the plasmid) are bordered at their ends by bidirectional transcription terminators (cf. example 1). On one side a fragment coding for the transcription terminator of the gene CYC1 (Tcyc1) (described by Osborne, B. I., and L. Guarente, 1989) and on the other side a fragment coding for the terminator of the gene ADH1 (Tadh1) (described by Irniger, S. et al., 1991.) are thus found. These two elements stop the transcription in a bidirectional manner. This prevents the extension of the transcription directed by Pgal1 or Padh1 beyond the restriction sites SalI/SacI and also protects the rY2H systems from the possible influence of transcription activities outside of the cassette of the reporter gene. The interference constructs were initially constructed on bacterial vectors and the restriction sites SalI and SacI were used for subcloning in replication and integration vectors of yeast (cf. below).

### EXAMPLE 3

### Construction of a Novel Integration Vector pRB2

A PCR fragment of 871 bp corresponding to the region+29 to +900 situated downstream of the stop codon TAA of the gene URA3 is generated by PCR by using the following oligonucleotides as primers: RB83 5′-AAAAAAGAGCTCTACTAAACTCACAAATTAGAGC-3′ (SEQ ID No. 17) and RB84 5′-AAAAAAGAATTCGCGGCCGCAAATATACTGGGGAACCAGTC-3′ (SEQ ID No. 18) (the sites EcoRI and SacI are underlined) and the strain FL100 of S. cerevisiae as matrix of the genomic DNA.

The PCR product is digested by EcoRI-SacI and cloned at the level of the sites EcoRI-SacI of the vector pFA6a-kanMX-PGAL1-3HA (described by Longtine, M. S., et al., 1998 and Wach A, et al., 1997 and of which the technical content as far as the construction of this vector is concerned is incorporated by reference to the present application). The vector thus obtained was called pRB12.

In parallel, a fragment of 769 bp corresponding to the region −223 to −992 situated upstream of the ATG of the gene URA3 is generated by PCR by using the following oligonucleotides as primers: RB85 5′-AAAAAACGTACGGCGGCCGCGATAAGGAGAATCCATACAAG-3′ (SEQ ID No. 19) and RB86 5′-AAAAAACGTACGTTTATGGACCCTGAAACCAC-3′ (SEQ ID No. 20) (the sites BsiWI are underlined) and the genomic DNA of the strain FL100 of S. cerevisiae as matrix. The PCR product is digested by the enzyme BsiWI and cloned at the level of the unique site BsiWI of pRB12. It has been possible to insert the fragment in both directions, a plasmid with the insert in the desired orientation (in which the two promoters Pgal1 are orientated in opposite directions) is selected by restriction profile analysis.

The sequence of this construct is verified by sequencing with the aid of the ‘Big Dye Terminator’ kit (Perkin-Elmer, Wellesley, Mass., USA).

The vector pRB2 contains two homologous fragments in the upstream and downstream regions, respectively, of the open reading frame of the gene URA3 of S. cerevisiae. A sequence inserted between these two fragments targets an integration at the level of the locus URA3, its genomic integration producing a deletion of 1104 bp, which eliminates the open reading frame of URA3 (cf. FIG. 3). This deletion of URA3 is near to the deletion published in the model ura3D0 (3), the difference with respect to ura3D0 being that 22 bp less of the region downstream of URA3 is removed during the integration of pRB2.

The gene GEA2 is situated upstream of UPA3 and it has been shown previously that the level of transcription and the size of the transcripts of the gene GEA2 are not affected by the deletion of ura3D0 (Brachmann, C. B., et al., 1998).
The gene TIM9 is located downstream of URA3 and is a gene essential to the yeast S. cerevisiae. A reduced rate of survival for the strains ura3D0 has not been reported; TIM9 proves to remain entirely functional in the strains ura3D0. Consequently, the deletion ura3D0 does not prove to affect the transcription of the adjacent genes. Consequently, the interference systems integrated with pRB2 will probably not affect nor will be affected by the transcription of GEA2 and TIM9. An advantage of the vectors pRB2 is that it is also possible to use them with mutant strains of URA3 like ura3-52 and ura3D0.

### EXAMPLE 4

### Subcloning of Different Inserts of the Interference Vectors in the Integration and Replication Vectors of pRB2 Yeast

The different interference and control systems (DI, nDI, UI, nUI and UDI) described in the examples 2 (cf. FIG. 2A to 2E) on pBluescript vectors are subcloned in the form of SalI-SacI fragments in the integration vector pRB2. In order to do that the different plasmids pRB16, pRB17, pRB18, pRB19 and pRB27 are digested by the enzymes SalI-SacI and the insert is subcloned in the integration vector pRB2 previously digested by the enzymes SalI-SacI. The different constructs obtained were called pRB31, pRB23, pRB24, pRB25 and pRB29 respectively (cf. table 2).

After integration of the interference systems into the genome, the reporter gene HIS3 is paired with respect to the genes GEA2 and TIM9.

TABLE 2
Vectors and interference strains.

| | In the | | | |
|---|---|---|---|---|
| | vector | | | Name of the strain |
| | pBluescript | In | In | once the construct |
| Constructs | KS+ | pLac33 | pRB2 | is integrated |
| nDI | pRB16 | — | pRB31 | yRB82 |
| DI | pRB17 | pRB20 | pRB23 | yRB48 |
| UI | pRB18 | pRB21 | pRB24 | yRB49 |
| nUI | pRB19 | — | pRB25 | yRB50 |
| UDI | pRB27 | pRB28 | pRB29 | yRB83 |

Abbreviations: nDI: nonsense downstream interference; DI: downstream interference; UI: upstream interference; nUI: nonsense upstream interference; UDI: upstream-downstream interference.

### EXAMPLE 5

### Construction of Transcriptional Interference Strains with Genomic Integration of Interference Vectors

5.1 Genomic Integration of Interference Vectors:

The culture media used are such as described by Gutherie, C. and G. R. Fink, 2002. For all the experiments, the cells are grown in a defined minimal medium YNB (Difco Laboratories, Detroit, USA)+2% glucose (Sigma-Aldrich, Lyon, France) at 30° C. In order to make up the auxotrophic markers, the required amino acids (Sigma-Aldrich, Lyon, France) are added.

The strain yCM15 (MAT a Δgal4 Δgal80 ura3-52 lys2-801 his3Δ200 trp1-Δ63 leu2 ade2-101) is used for the constructs of strains with genomic integration. The strain yCM15 was constructed starting from the strain PCY2 (described by Chevray P M and Nathans D., 1992), in which the reporter cassette has been eliminated.

The strain PCY2 (MAT α Δgal4 Δgal80 URA3::GAL1-lacZ lys2-801 ade2-101 trp1-Δ63 his3-Δ200 leu2) is the result of a crossing between the strain YPH499 (MAT a ura3-52 lys2-801 ade2-101 trp1-Δ63 his3-Δ200 leu2-Δ1; ATCC 76625, 204679; (Sikorski and Hieter, 1989)) (accessible especially from the American Type Culture Collection (ATCC) (Manassas, Va., USA), under the number 204679) with the strain GGY1::171 (MAT α Δgal4 Δgal80 leu2 his3 URA3::GAL1-lacZ) (Gill and Ptashne, 1987).

The strain GGY1:171 was constructed by the integration of the cassette URA3::GAL1-lacZ (Yocum et al., 1984), which carries the reporter gene lacZ under the control of the promoter of the gene GALL activated by UAS_{G}, into the strain GGY1 (MAT α Δgal4 Δgal80 leu2 his3 (Gill and Ptashne, 1987)). The strain GGY1 is itself the result of a crossing between the strain DBY745 (MAT α Δgal4 Δgal80 leu2-3,112 ade1-100 ura3-52; (Graham and Chambers, 1996; Ferreiro et al., 2004; Nagarajan and Storms, 1997; Dormer et al., 2000) and the strain YM709 (Pearlberg, J., 1995).

In place of the strain PCY2, any strain of yeast S. cerevisiae having the genotype (gal4-gal80-trp1-his3-leu2-ura3-) can be used. For example, in place of the strain PCY2, the strain Y187 (described by Harper, J. W. et al., 1993 and accessible especially from the American Type Culture Collection (ATCC) (Manassas, Va., USA), under the reference number 96399) can be used.

Table 3 shows other examples of strains which can be substituted for the strain PCY2. Their designations, a short description of these strains and their reference numbers with the American Type Culture Collection (ATCC) (Manassas, Va., USA), a body from which these strains are accessible, are shown.

In order to construct the strain yCM15, 10⁶ to 10⁷ cells of a clone of the strain PCY2 are suspended in sterile water and streaked on an agar plate of YNB medium+2% glucose made up by the amino acids lys+ade+his+ura+trp+leu. The agar plate also contains 5-fluoroorotic acid (5-FOA) at a concentration of 1 mg/ml; 5-FOA is toxic for the cells expressing the gene URA3. Consequently, with these plates containing 5-FOA, the mutants of the strain PCY2 which have lost the reporter cassette URA3::GAL1-lacZ are selected (Boeke et al., 1987). The absence of the reporter cassette in the mutants obtained is verified by testing the activity of the reporter lacZ. The mutants are then transformed with the plasmid pCL1 (Fields, S., and O, Song. 1989) coding for the transcription factor GAL4p which is a very strong activator of the promoter GALL and the lacZ activity is measured (Breeden, L. and K. Nasmyth, 1985). A mutant resistant to 5-FOA and without lacZ activity is selected and called yCM15.

TABLE 3
Strain which can be substituted for the strain PCY2

| | | ATCC |
|---|---|---|
| Designation | Description | reference |
| CTY10-5D | Saccharomyces cerevisiae | 201449 |
| | Hansen, teleomorph (budding | |
| | yeast) | |
| PJ69-4A | Saccharomyces cerevisiae | 201450 |
| | Hansen, teleomorph (budding | |
| | yeast) | |
| JC981 | Saccharomyces cerevisiae | 204096 |
| | Hansen, teleomorph (budding | |
| | yeast) | |
| JC993 | Saccharomyces cerevisiae | 204097 |
| | Hansen, teleomorph (budding | |
| | yeast) | |
| Y166 | Saccharomyces cerevisiae | 96397 |
| | Hansen, teleomorph (budding | |
| | yeast) | |
| YAS1760 | Saccharomyces cerevisiae | MYA-2200 |
| | Hansen, teleomorph (budding | |
| | yeast) | |
| YAS1924 | Saccharomyces cerevisiae | MYA-2210 |
| | Hansen, teleomorph (budding | |
| | yeast) | |
| Y190 | Saccharomyces cerevisiae | MYA-320 |
| | Hansen, teleomorph (budding | |
| | yeast) | |

The integration vectors pRB31, pRB23, pRB24, pRB25 and pRB29 are linearized by digestion with the enzyme DrdI. The strain yCM15 is transformed independently by 1 μg of linearized DNA of each of the integration vectors by using the method of lithium acetate transformation (such as described by Gietz, R. D. and A. Sugino, 1988) and the cells are grown on a depleted histidine medium (YNB (Difco Laboratories, Detroit, USA)+2% glucose (Sigma-Aldrich, Lyon, France) completed by the amino acids lys+ade+trp+leu and uracil (Sigma-Aldrich, Lyon, France). The auxotrophic clones for histidine are isolated and the integration of the reporter cassettes at the level of the locus URA3 is verified by colony PCR (described by Gutherie, C et al, 2002) by using internal and external primers specific for each junction (see table 4).

TABLE 4

| | upstream URA3 junction | | | downstream URA3 junction | | |
|---|---|---|---|---|---|---|
| | | | PCR | | | PCR |
| | primer | primer | product | primer | primer | product |
| vector | 1 | 2 | size (bp) | 1 | 2 | size (bp) |
| pRB23 | RB103 | RB61 | 1183 | RB68 | RB109 | 1252 |
| pRB24 | RB103 | RB68 | 1155 | RB66 | RB109 | 1395 |
| pRB25 | RB103 | RB38 | 1294 | RB66 | RB109 | 1395 |
| pRB29 | RB103 | RB68 | 1155 | RB68 | RB109 | 1253 |
| pRB31 | RB103 | RB61 | 1183 | RB38 | RB109 | 1389 |

The sequences of the primers used are the following:

| | |
|---|---|
| RB38: | |
| 5′-GGGGTAATTAATCAGCGAAGCGATG-′3 | (SEQ ID NO. 21) |
| RB61: | |
| 5′-CGATGTATGGGTTTGGTTG-′3 | (SEQ ID No. 22) |
| RB66: | |
| 5′-TCTTGCGAGATGATCCCGC-′3 | (SEQ ID No. 23) |
| RB68: | |
| 5′-TAAGCGTATTACTGAAAGTTCC-′3 | (SEQ ID No. 24) |
| RB103: | |
| 5′-GCAATGAAAGAGCAGAGCGAGAG-′3 | (SEQ ID No. 25) |
| RB109: | |
| 5′-GTTATCAGATATTATCAGGTGGGAA-′3 | (SEQ ID No. 26) |

The strains obtained by the independent transformation of the strain yCM15 by each of the integration vectors pRB31, pRB23, pRB24, pRB25, pRB29, and of which the integration of each of the reporter cassettes at the level of the locus URA3 has been demonstrated, were called yRB82, yRB48, yRB49, yRB50, yRB83 respectively (cf. table 2).

5.2 Activation of Integrated Interference Constructs:

In order to evaluate the activity of interference systems, the reporter strains obtained in example 5.1 are transformed with two pairs of plasmids. The first pair is used in order to study the response of interference systems with respect to powerful activation of Pgal1 owing to the expression of the GAL4p transcription factor of complete length (Fields, S. and O. Song, 1989). The second pair of plasmids allows the expression of the activation domain (AD) and of the DNA-binding domain (DBD) of GAL4p separately and serves as a negative control because the single distinct domains cannot activate Pgal1.

Thus the strains obtained in example 5.1 (strains yRB82, yRB48, yRB49, yRB50, yRB83) are cotransformed by the centromeric plasmid pCL1 coding for the GAL4p transcription factor (described by Fields, S., and O, Song. 1989) and the plasmid pFL39 without a centromeric insert (described by Bonneaud N, et al., 1991). These plasmids being replicated in yeast carry the gene LEU2 or TRP1 as a selection marker.

The transformants are selected on a YNB medium (Difco Laboratories, Detroit, USA)+2% glucose (Sigma-Aldrich, Lyon, France) completed by the amino acids lys+ade+his and uracil (Sigma-Aldrich, Lyon, France).

The transformants obtained are respectively called nDI+, DI+, UI+, nUI+ and UDI+.

The negative controls are obtained by cotransforming the strains described in example 5.1 (strains yRB82, yRB48, yRB49, yRB50, yRB83) by the vectors pTAL20 and pDBT. The vectors pTAL20 and pDBT allow the expression of the activation domain (AD) of GAL4p and of the DNA-binding domain (DBD) of GAL4p (these vectors are described by Navarro, P., et al., 1997).

The transformants thus obtained are respectively called nDI−, DI−, UI−, nUI− and UDI−.

### EXAMPLE 6 STUDY OF THE GROWTH OF YEAST HAVING A TRANSCRIPTIONAL INTERFERENCE SYSTEM INTEGRATED INTO THEIR GENOME

The cell growth of the transformants is studied by a drop evaluation method. In order to do that, the cells are deposited on agar plates containing histidine and incubated for one night at 30° C. Then, the cells are suspended in water at approximately 10⁶ cells/ml and dilution series of a factor 10 are prepared. Drops of 5 μl of each dilution are withdrawn by pipette and they are deposited on predried agar plates comprising the desired medium. The plates are incubated at 30° C.

The cell growth of the interference transformants is thus evaluated on histidine-depleted media. A competitive inhibitor of the protein HIS3p (3-amino-1,2,4-triazole (3-AT)) is optionally added to the culture medium at growing concentrations in order to observe differences at the level of the level of activity of the reporter gene HIS3 between the different interference strains (FIG. 4). The media containing the inhibitor are prepared starting from a stock solution at the concentration of 1M of the competitive inhibitor 3-amino-1,2,4-triazole (3-AT; Sigma) in water of Millipore quality. The agar is cooled to 60° C. before the addition of the 3-AT at the desired concentrations. The different culture media tested are a minimum medium (YNBG (Difco Laboratories, Detroit, USA)+2% glucose (Sigma-Aldrich, Lyon, France) completed by the amino acids lys+ade and uracil (Sigma-Aldrich, Lyon, France) without 3-AT or with 3-AT at concentrations of 8, 16, 22, 32 mM. A positive control is also carried out (culture medium with histidine and without 3-AT). The plates are incubated at 30° C. for 48 to 72 hours. Some of the results are presented in FIG. 4A to 4E.

In the presence of GAL4p (+strains), the greatest reduction in the cell growth is observed for the UI+ transformants. The UI cells expressing GAL4p cannot grow in the absence of histidine whereas the cells carrying the control AD/DBD (UI− transformants) grow normally at concentrations of 3-AT higher than 32 mM. Neither GAL4p nor the control AD/DBD inhibit the cell growth of the control transformants nUI+ and nUI−, which shows that the inhibition of growth of the cells UI+ expressing GAL4p is due to the activation of the transcription of Pgal1 directed toward Padh1.

In the DI+ cells, the expression of GAL4p does not affect the cell growth when the 3-AT inhibitor is absent (cf. FIG. 4B), but the growth is totally inhibited when 3-AT is present at 8 mM or at higher concentrations (cf. FIG. 4C to 4E). On the contrary, the cell growth of the control transformants DBD/AD (DI− transformants) is not affected even at concentrations of 3-AT of 32 mM (cf. FIG. 4E). Consequently, the transcription directed by Pgal1 of the antisense strand of the gene HIS3 significantly increases the level of auxotrophy for the histidine of the DI+ cells.

Surprisingly, GAL4p also inhibits the cell growth in the nDI+ transformants (the growth is inhibited when the 3-AT is present at 8 mM or at higher concentrations (cf. FIG. 4C to 4E). In these cells, the transcription directed by Pgal1 is directed toward Tadh1 (cf. FIG. 2D); it is not expected that it interferes with the expression of HIS3. The inhibition of growth is due to the specific activation of Pgal1 because inhibition of growth is not observed when the control AD/DBD is present in the nDI− cells (cf. FIG. 4B to 4E).

In the presence of GAL4p, the UDI+ transformants show a level of inhibition of growth similar to that of DI+ cells. The control DBD/AD does not affect the growth of the UDI− cells (cf. FIG. 4B to 4E).

We can conclude that in the strains UI+, DI+ and UDI+, the activation due to GAL4p of the promoter Pgal1 leads to a significant inhibition of cell growth and that this inhibition is due to reduced activity of the reporter HIS3. The inhibition of growth is slightly greater in the UI cells than in the DI and UDI cells.

The reduction induced by GAL4p of the activity of HIS3 thus significantly slows down the cell growth; however, it does not inhibit it completely. Although under interference conditions colonies on the plates are not detected for the first 6-7 days of incubation, minuscule colonies become visible after longer incubation times.

We have observed that the interference is more powerful with the UI system than with the DI, UDI or nDI systems, which can arise from the molecular mechanism generating the transcription interference. Detailed studies of upstream interference have shown that upstream promoter activation reduces the activity of the downstream promoter by removing the activation factors from their sites of linkage at the level of the downstream promoter (Greger, I. H. et al., 2000, Greger, I. H. et al., 1998, Valerius, O. C. et al., 2002, Callen B P et al., 2004). The promoter generating interference being nearer to the promoter of the reporter gene in the UI construct, the interference can be more efficacious in the UI than in the DI construct.

It is also possible that downstream interference arises from a different molecular mechanism. In the system DI, RNA polymerases transcribing in a convergent manner can enter into collision and this can lead to a stop in transcription and to silencing of the gene HIS3. A frontal collision of RNA polymerases could be a less efficacious means of silencing HIS3 than the removal of transcription activation factors of Padh1. In addition, it is possible that in the DI system, the activation of Padh1 inactivates Pgal1 by transcription interference, which can also explain why the UDI system is not superior either to the UI system or to the DI system.

Surprisingly, the applicant has observed that downstream transcription interference also exists when Pgal1 is inserted paired and downstream with respect to the reporter cassette HIS3 (nDI construct). The level of transcription of a gene depends on the power of the promoter as well as on the quality of the transcription stop signal (Wahle, E., and U. Ruegsegger, 1999). Pgal1 being inserted between the ORF of HIS3 and its transcription terminator Tadh1, activation of Pgal1 could interfere with an appropriate stopping of the transcript of HIS3 in the construct nDI, which thus reduces the activity of HIS3p in the cells.

### EXAMPLE 7

### Subcloning of the Different Inserts of the Interference Vectors in the Vector pLac33

In order to test whether the transcriptional interference systems are functional when they are present on a plasmid and not integrated at the genomic level, the different interference systems (DI, UI, and UDI) described in example 2 (cf. FIG. 2A to 2E) on pBluescript vectors are subcloned in the form of SalI-SacI fragments in the integration vector pLac33. This vector contains the gene URA3 of yeast as a selection marker and has been described by Gietz, R. D., and A. Sugino. 1988. In order to do that, the different plasmids pRB27, pRB17, pRB18 are digested by the enzymes SalI-SacI and the insert is subcloned in the replicative vector pLac33 previously digested by the enzymes SalI-SacI. The different constructs obtained were called pRB28, pRB20, pRB21 respectively.

### EXAMPLE 8

### Study of the Growth of Yeast Having a Nonintegrated Transcriptional Interference System in their Genome

It has also been studied whether the transcription interference constructs UI, DI and UDI are also functional when they are present on a plasmid and not integrated at the genomic level. In order to do that, the vectors derived from the plasmid pLac33 carrying the different transcriptional interference constructs and described in example 7 were used in order to transform independently the strain yCM15.

The strain yCM15 is cotransformed independently by 0.3 μg of DNA of each of the vectors pRB28, pRB20 or pRB21 (cf. example 7) associated with the vectors pCL1 and pFL39. The strain yCM15 is thus transformed independently by three groups of three plasmids (pRB28+pCL1+pFL39, or pRB20+pCL1+pFL39 or pRB21+pCL1+pFL39). The centromeric plasmid pCL1 codes for the GAL4p transcription factor (described by Fields, S., and O, Song. 1989) and the plasmid pFL39 is a plasmid without centromeric insert (described by: Bonneaud N, et al., 1991). These yeast replication plasmids carry the gene LEU2 or TRP1 as a selection marker.

The strain yCM15 is also cotransformed independently by 0.3 μg of DNA of each of the vectors pRB28, pRB20 or pRB21 (cf. example 7) associated with the vectors pTAL20 and pDBT (allowing the expression of the activation domain (AD) of GAL4p and of the DNA-binding domain (DBD) of GAL4p (these vectors are described by Navarro, P., et al., 1997)). The strain yCM15 is thus transformed independently by three groups of three plasmids (pRB28+pTAL20+pDBT, or pRB20+pTAL20+pDBT or pRB21+pTAL20+pDBT).

The cell growth of each of these triple transformants is studied on agar plates with and without histidine and, for the plates without histidine, without or with growing concentrations of 3-AT, as described in example 6.

The results obtained with the interference constructs based on nonintegrated plasmids are similar to those obtained for the integrated interference constructs (cf. FIG. 5A to 5D). Thus when the medium is depleted in histidine and in 3-AT, the UI cells expressing GAL4p (triple transformants pRB21+pCL1+pFL39) show very strongly reduced growth compared with the UI cells not expressing active GAL4p (triple transformants pRB21+pTAL20+PDBT). The level of inhibition of growth of UI is not, however, as great as that observed with the integrated version of the construct UI (cf. example 6).

The DI cells (triple transformants pRB20+pCL1+pFL39 (expressing an active GAL4p) or pRB20+pTAL20+PDBT (not expressing an activeGAL4p)) and UDI (triple transformants pRB28+pCL1+pFL39 ((expressing an active GAL4p) or pRB28+pTAL20+pDBT (not expressing an active GAL4p) grow quasi-normally whether they do or do not express active GAL4p.

When the medium contains 3-AT at 8 mM or at a higher concentration, the cells expressing GAL4p and transformed by the construct UI, DI or UDI do not grow any more at all (cf. FIGS. 5C and 5D). This result contrasts with those obtained for the negative control cells (not expressing active GAL4p (AD/DBD)) and transformed by the interference plasmids whose cell growth is not affected (cf. FIGS. 5C and 5D).

The applicant concludes that versions based on plasmids of UI, DI and UDI are functional but slightly less sensitive to activation of Pgal1 than the corresponding integrated versions of the interference constructs.

### EXAMPLE 9

### Independence of the Transcription Interference Systems with Respect to the Genetic Background of the Host Strain

In order to study whether the activity of the transcription interference systems depends on the genetic background of the host strain, constructs based on plasmids (such as described in example 7) have also been evaluated in a second strain of yeast: the strain yRB31. The strain yRB31 (MAT a ura3-52 his3-Δ200 ade2-101 trp1-901 leu2-3,112 met-Δgal4 Δgal80) is constructed by eliminating the cassette URA3::GAL1 (UAS)-GAL1 (TATA)-lacZ of the strain Y187 (the strain Y187 has been described Harper, J. W. et al., 1993 and is accessible especially from the American Type Culture Collection (ATCC) (Manassas, Va., USA), under the reference number 96399).

In order to construct the strain yRB31, 10⁶ to 10⁷ cells of a clone of the strain Y187 are suspended in sterile water and streaked on an agar plate of YNB medium+2% glucose completed by the amino acids met+ade+his+ura+trp+leu. The agar plate also contains 5-fluoroorotic acid (5-FOA) at a concentration of 1 mg/ml; 5-FOA is toxic for the cells expressing the gene URA3. Consequently, with these plates containing 5-FOA, mutants of the strain Y187 which have lost the reporter cassette URA3::GAL1(UAS)-GAL1(TATA)-lacZ are selected (Boeke et al., 1987). The absence of the reporter cassette in the mutants obtained is verified by testing the activity of the reporter lacZ. The mutants are transformed with the plasmid pCL1 (Fields S. and O. Song, 1989) coding for the GAL4p transcription factor which is a very strong activator of the promoter GAL1 and the lacZ activity is measured (Breeden L. and K. Nasmyth, 1985). A mutant resistant to 5-FOA and without lacZ activity is selected and called yRB31.

The strain yRB31 was transformed in the same manner as described in example 8 for the strain yCM15. The transformants obtained were studied as far as their cell growth is concerned in the same fashion as described in example 8. No difference was observed between the strains yCM15 and yRB31 as far as the activity of the interference plasmids is concerned.

### EXAMPLE 10

### Induction of the Expression of the UI and DI Interference Systems by the Protein-Protein Interaction and Effect on the Inhibition of the Cell Growth

10.1: Induction of the Expression of Interference Systems by the Interaction of the Protein Fe65 with the Previously Identified Protein APP

It was studied whether a protein-protein interaction previously identified between the proteins Fe65 and APP (McLoughlin D M and Miller C C, 1996; Fiore F et al., 1995; Zambrano N et al., 1997) is capable of sufficiently inducing a transcription interference construct in order to inhibit the cell growth of yeast.

The integrated versions of the constructs UI and DI are used. The fusion proteins AD-Fe65 and DBD-AppCt are expressed under the control of the strongly active truncated ADH1 promoter (promoter described by Ruohonen L, et al., 1995).

The plasmid pRB34 allows the expression of the fusion protein AD-Fe65 under the control of a strongly active truncated ADH1 promoter.

The construction of the plasmid pRB34 was carried out in several steps. In a first step a part of the reading frame of the gene Fe65 is cloned in the vector pGAD424 (BD Biosciences Clontech, (Palo Alto, Calif., USA)). A PCR fragment of 1694 bp corresponding to the amino acids 152 to 708 of the gene Fe65 (Genebank accession number: BC010854) is generated by PCR by using the following oligonucleotides as primers: RB250 5′-AGATCGAATTCAAGGCGGCCGGGGAGGCCGAGG-3′ (SEQ ID No. 27) and RB251 5′-GCAGGTCGACTCATGGGGTATGGGCCCCCAGC-3′ (SEQ ID No. 28) (the sites EcoRI and SalI are underlined) and a bank plasmid DNA of cDNA of commercial human brain as matrix (BD Biosciences Clontech, (Palo Alto, Calif., USA)). The PCR product is digested by the enzymes EcoRI-SalI and cloned at the level of the sites EcoRI-SalI of the vector pGAD424. The vector thus obtained is called pRBSG11. The sequence of this construct is verified by sequencing with the aid of the ‘Big Dye Terminator’ kit (Perkin-Elmer, Wellesley, Mass., USA).

In a second step, a partial digestion of the plasmid pRBSG11 is carried out with the enzyme SphI and the fragment coding for the weakly active truncated promoter ADH1, the fusion protein AD-Fe65 and the terminator ADH1 is inserted in the site SphI of the centromeric plasmid pFL36 (described by Bonneaud N, et al., 1991); this allows the plasmid pRBIM54 to be obtained. The obtainment of the good construct is verified by restriction analysis. The fragment can be inserted in both directions and a clone having the desired orientation, in which the ADH1 promoter is near to the unique site SacI of the plasmid pFL36, is selected. In a third step, a fragment coding for the strongly active truncated ADH1 promoter is amplified by PCR starting from the genomic DNA of the strain FL100 with the primers RB136 (5′-TTGTAAAACGACGGCCAGTGAATTCCGTACGATATCCTTTTGTTGTTTCCGGGTG-3′) (SEQ ID No. 29) and RB137 (5′-AGTTGATTGTATGCTTGGTATAGC-3′) (SEQ ID No. 30). By using the method of cloning by homologous recombination (GAP repair) (DeMarini et al., 2001; Orr-Weaver et al., 1983), the PCR fragment is used in order to replace the weakly active truncated ADH1 promoter by the strongly active truncated ADH1 promoter in the plasmid pRBIM54 previously digested by the enzyme SacI. The homologous recombination is carried out in the strain yCM15. The novel plasmid constructed is verified by restriction analysis and is called pRB34. The sequence of this plasmid is shown in SEQ ID No. 31.

The plasmid pRB35 allows the expression of the fusion protein DBD-AppCt under the control of a strongly active truncated of ADH1. The construction of the plasmid pRB35 was carried out in several steps.

In a first step, the carboxy-terminal part of the reading frame of the gene HSAFPA4 (App, accession number EMBL Y00264) is cloned in the vector pGBT9 (BD Biosciences Clontech, (Palo Alto, Calif., USA)). A fragment of 143 bp coding for the amino acids 650 to 695 of the gene HSAFPA4 is obtained by hybridization of two phosphorylated single-strand oligonucleotides of sequence: RB252 5′-AATTCAAGAAACAGTACACATCCATTCATCATGGTGTGGTGGAGGTTGACGCCGCTGT CACCCCAGAGGAGCGCCACCTGTCCAAGATGCAGCAGAACGGCTACGAAAATCCAACC TACAAGTTCTTTGAGCAGATGCAGAACTAGG-3′ (SEQ ID No. 32) and RB253 of sequence: 5′-TCGACCTAGTTCTGCATCTGCTCAAAGAACTTGTAGGTTGGATTTTCGTAGCCGTTCT GCTGCATCTTGGACAGGTGGCGCTCCTCTGGGGTGACAGCGGCGTCAACCTCCACCAC ACCATGATGAATGGATGTGTACTGTTTCTTG-3′ (SEQ ID No. 33). In order to do that, the two single-strand oligonucleotides are mixed at equimolar concentrations in water. The mixture is heated at 90° C. for 5 min in a heating block (heat block) and then the block is allowed to cool to ambient temperature (approximately 2 hours). The double-stranded fragment thus obtained has compatible cohesive extremities EcoRI and SalI, respectively, at its ends. This allows the insertion of this double-stranded fragment (corresponding to the carboxy-terminal part of HSAFPA4) in the vector pGBT9 (BD Biosciences Clontech). The vector thus obtained allows the expression of the fusion protein between GAL4 DBD and the amino acids 650 to 695 of App (DBD-AppCt) and is called pSG14. The sequence of this construct is verified by sequencing with the aid of the ‘Big Dye Terminator’ kit (Perkin-Elmer, Wellesley, Mass., USA).

In a second step, the plasmid pSG14 is partially digested with the enzyme SphI. Then the fragment coding for the weakly active truncated ADH1 promoter, the fusion DBD-AppCt and the ADH1 terminator is inserted in the vector pFL39 (described by Bonneaud N, et al., 1991) previously digested by the enzyme SphI. The resulting plasmid is called pIM55. The obtainment of the good construct is verified by restriction analysis. The fragment can be inserted in both directions and a clone having the desired orientation, in which the ADH1 promoter is near to the unique site SacI of the plasmid pFL36, was selected.

The plasmid pRB35 is then obtained by exchanging the weakly active truncated ADH1 promoter of pIM55 by a strongly active truncated ADH1 promoter. The same PCR fragment as that used for the construction of pRB34 is introduced into pIM55 previously digested by the enzyme SphI by using the same method of GAP repair. The plasmid constructed is verified by restriction analysis and is called pRB35. The sequence of this plasmid is shown in SEQ ID No. 34.

Firstly, the activity of the constructs was verified. In order to do that, the reporter strain Y187 (the strain Y187 has the gene URA3 placed under the control of the promoter Pgal1 and was described by Harper, J. W. et al., 1993 and is accessible especially from the American Type Culture Collection (ATCC) (Manassas, Va., USA), under the reference number 96399) is transformed by three pairs of plasmids. These pairs are the following: pRB34+pRB35, pRB34+pDBT and pTAL20+pRB35. Two clones cotransformed by each of these pairs of plasmids are selected. Their capacity to grow on minimum medium with and without uracil is studied. It is shown that the six corresponding clones are capable of growing on minimum medium in the presence of uracil (cf. FIG. 6A). However, in the absence of uracil, only the two clones cotransformed by the plasmids pRB34+pRB35 are capables of growing (cf. FIG. 6A). This shows that the gene URA3 is activated in the strain Y187 and this with the aid of the interaction between AD-Fe65 and DBD-AppCt reconstituting an active transcription factor GAL4p capable of activating the promoter Pgal1. The activity of the constructs used is thus demonstrated.

The strains yRB48 (DI system) and yRB49 (UI system) are next cotransformed, each by the pair of plasmids pRB34 and pRB35, allowing the expression of the two hybrid proteins AD-Fe65 and DBD-AppCt. The strains yRB48 (DI system) and yRB49 (UI system) are also cotransformed, each by the pairs of plasmids pRB34+PDBT and pTAL20+pRB35 as controls. Two clones cotransformed by each of these pairs of plasmids are selected. The cell growth of each of the two clones of these double transformants is studied on agar plates with and without histidine and on plates without histidine and with or without growing concentrations of 3-AT, as described in example 6.

As shown in FIG. 6B to 6E, the interaction between the fusion proteins AD-Fe65 and DBD-AppCt inhibits the cell growth of the strains carrying the interference constructs UI and DI although the cells expressing the fusion protein AD-Fe65 and the domain DBD or the fusion protein DBD-AppCt and the domain AD grow normally. In this experiment, the differences at the level of the cell growth are detected when 3-AT is added to concentrations higher than 15 mM.

It can be concluded that transcription interference induced by double-hybrid interaction can be easily detected with the rY2H UI and DI systems.

10.2: Induction of the Expression of Interference Systems by the Interaction of the Protein p53 with the Protein mdm2 Previously Identified:

It has also been studied whether another previously identified protein-protein interaction between the proteins p53 and mdm2 (Oliner J D et al., 1993; Chen J et al., 1993; Momand J et al., 1992) is capable of sufficiently inducing a transcription interference construct in order to inhibit the cell growth of yeast. In order to do that, the integrated version of the construct UI is used. The fusion proteins DBD-mdm2 and VP16-p53(1-55) are expressed under the control of the complete promoter of the gene ADH1 starting from 2μ plasmids.

In order to construct the vector pVP16-p53Nt, the open reading frame coding for the 55 amino acids of the amino end of the human p53 protein (genebank reference: BC003596) is generated by hybridization of two phosphorylated single-strand oligonucleotides: RB200 5′-GGCCGCAGTGAACCATTGTTCAATATCGTCCGGGGACAGCATCAAATCATCCATTGCT TGGGACGGCAAGGGGGACAGAACGTTGTTTTCAGGAAGTAGTTTCCATAGGTCTGAAA ATGTTTCCTGACTCAGAGGGGGCTCGACGCTAGGATCTGACTGCGGCTCCTCCATCTG-′3 (SEQ ID No. 35) and RB201 5′-GATCCAGATGGAGGAGCCGCAGTCAGATCCTAGCGTCGAGCCCCCTCTGAGTCAGGAA ACATTTTCAGACCTATGGAAACTACTTCCTGAAAACAACGTTCTGTCCCCCTTGCCGT CCCAAGCAATGGATGATTTGATGCTGTCCCCGGACGATATTGAACAATGGTTCACTGC-3′ (SEQ ID No. 36) (the sites NolI and BamHI are underlined). In order to do that, the two complementary single-strand oligonucleotides are mixed in equimolar concentrations in water. The mixture is heated at 90° C. for 5 min in a heating block (heat block) and then the block is allowed to cool to ambient temperature (approximately 2 hours). The double-stranded fragment thus obtained has compatible cohesive extremities NotI and BamHI, respectively, at the ends.

Then a fragment ligation is carried out in the vector pVP16 (Vojtek and Hollenberg, 1995; Vojtek et al., 1993) digested previously by the enzymes NotI-BamHI. The sequence of this construct is verified by sequencing with the aid of the ‘Big Dye Terminator’ kit (Perkin-Elmer, Wellesley, Mass., USA).

In order to construct the vector pDTB-mdm2, the gene coding for the entire Mdm2 human protein (genebank reference: NM_{—}002392) is amplified by PCR by using the vector pCR3-mdm2fl (Sigalas et al., 1996) as matrix and the following oligonucleotides: RB202 (5′-CCCGGGAATTCAGATCCATATGTGCAATACCAACATGTCTGTAC-3′) (SEQ ID No. 37) and RB203 (5′-ACTTAGAGCTCTAGGGGAAATAAGTTAGCACAATC-3′) (SEQ ID No. 38) (the sites EcoRI and SacI are underlined). The PCR product is digested by the enzymes EcoRI and SacI and cloned at the level of the sites EcoRI and SacI in the vector PDBT (Navarro et al., 1997). The sequence of this construct is verified by sequencing with the aid of the ‘Big Dye Terminator’ kit (Perkin-Elmer, Wellesley, Mass., USA).

The activity of the constructs obtained was verified. In order to do that, the reporter strain Y187 (the strain Y187 has the gene URA3 placed under the control of the promoter Pgal1 and has been described by Harper, J. W. et al., 1993 and is accessible especially from the American Type Culture Collection (ATCC) (Manassas, Va., USA), under the reference number 96399) is transformed by three pairs of plasmids. These pairs are the following: PDBT-mdm2+pVP16-p53Nt, pVP16-p53Nt+pDBT and pTAL20+pDBT-mdm2. Two clones cotransformed by the two plasmids are selected for each of these pairs of plasmids. Their capacity to grow on minimum medium with and without uracil is studied. It is shown that the six corresponding clones are capable of growing on minimum medium in the presence of uracil (cf. FIG. 6F). However, in the absence of uracil, only the two clones cotransformed by the plasmids pDBT-mdm2+pVP16-p53Nt are capable of growing. This shows that the gene URA3 is activated in the strain Y187 and this with the aid of the interaction between DBD-mdm2 and VP16-p53(1-55) reconstituting an active transcription factor capable of activating the promoter Pgal1. The activity of the constructs used is thus demonstrated.

The strain yRB49 (UI system) is next cotransformed by the pair of plasmids pDBT-mdm2+pVP16-p53Nt allowing the expression of the two hybrid proteins DBD-mdm2 and VP16-p53(1-55). The strain yRB49 (UI system) is likewise cotransformed by the pair of plasmids pDBT-mdm2+pTAL20 as controls. Two clones cotransformed by the two plasmids are selected for each of these two pairs of plasmids. The cell growth of each of the two clones of these double transformants is studied on agar plates with and without histidine and on plates without histidine and with or without increasing concentrations of 3-AT, as described in example 6. As shown in FIG. 6G, the interaction between the fusion proteins DBD-mdm2 and VP16-p53(1-55) inhibits the cell growth of the strains carrying the interference constructs UI although the cells expressing the fusion protein DBD-mdm2 and the domain AD grow normally.

In this experiment, the differences at the cell growth level are detected in the presence of a concentration of 3-AT equal to or higher than 5 mM.

It can be concluded that transcription interference induced by double-hybrid interaction can be easily detected with the rY2H UI systems.

### EXAMPLE 11

### Application of the rY2H UI Transcriptional Interference System to Screening for Protein-Protein Interaction Inhibitors

11.1: Calibration of the Test for Restoration of Yeast Growth on Solid Media

A yeast strain containing an rY2H UI transcriptional interference system integrated into its genome (yRB49) was used to isolate protein-protein interaction inhibitors by means of a test for restoration of growth.

To carry out this test, a yeast strain containing the rY2H UI transcriptional interference system, coupled to a specific protein-protein interaction, is plated out onto solid medium containing 3-AT. Drops of solutions of chemical compounds are deposited onto the inoculated dish. The diffusion of the chemical compounds in the agar creates a concentration gradient around the deposit zone. The products capable of inhibiting the protein-protein interaction allow cell growth, which is reflected by a growth restoration halo surrounding the deposit zone.

In order to determine the conditions for the development of such a test, a series of preliminary experiments was carried out. The first step was thus intended to evaluate the amount of cells to be deposited onto the Petri dishes and the second step was intended to evaluate the minimum concentration of 3-AT in the culture medium for inhibiting the cell growth.

First of all, yeasts containing an integrated version of the UI system (yRB49) were cotransformed with the plasmids pRB34 and pRB35 in order to place the transcriptional interference system under the control of a protein-protein interaction couple, Fe65/App. These yeasts were then cultured in liquid medium containing histidine. As soon as the optical density (OD) of the culture reached the value of 3 (3×10⁷ cells per ml), the cells were centrifuged for 10 minutes at 3000 rpm, washed twice with sterile water and resuspended at an OD=0.1 or 0.01. 10 ml of this suspension were plated out uniformly over the entire surface of the solid culture medium poured beforehand into 120 mm square Petri dishes. After one minute, the liquid was removed by suction. The dishes were dried for 15 minutes under a laminar flow hood and then incubated at 30° C. for 48 hours.

FIG. 8A shows that the optimal inoculation of the dishes was achieved with the lowest dilution, i.e. OD=0.01 (right-hand dish).

For the purpose of determining the minimum concentration of 3-AT capable of inhibiting the growth of the yeast, the yRB49 strain was transformed as above with the plasmids pRB34 and pRB35 (Fe65/App) or with the control plasmids pTAL20/pDBT (not expressing protein-protein interactions). The two transformed strains were plated out onto medium either containing histidine or free of histidine and supplemented with 0, 0.5 or 1 mM of 3-AT. As shown in FIG. 8B, the strain carrying the Fe65/App interaction exhibits a very retarded growth on medium containing 1 mM of 3-AT, compared with that containing the control plasmids, the growth of which is not affected under the same conditions.

In the same experiment, in order to rapidly test the toxicity of the molecules in solution in this growth restoration test, two molecules were selected randomly from a library of 10 000 compounds. After having been dissolved at 10 mM in DMSO, 1 μl of each was deposited onto each dish, as was 1 μl of DMSO. Neither of these two molecules is capable of restoring the yeast growth. However, they produce a growth inhibition disk around the molecule deposit zone, related to the toxic effects of these molecules at high concentration (FIG. 8B).

Addition of histidine to the medium corrects the strain's histidine synthesis deficiency by freeing it of the transcriptional interference system. In order to confirm that the experimental conditions described above make it possible to detect restoration of yeast growth, the yRB49 strain cotransformed with the pair of plasmids pRB34 and pRB35 (Fe65/App interaction) was plated out onto the above medium free of histidine and supplemented with 1 mM 3-AT, and then increasing amounts of uracil, adenine, leucine or histidine were deposited at the center of each dish.

As shown in FIG. 8C, only histidine is capable of restoring the growth of the yeast around the deposit, and in a manner dependent on its concentration.

11.2: Detection of Yeast Growth Restoration Induced by a Protein-Protein Interaction Inhibitor

The experimental conditions reported above were used on a large scale in order to screen a collection of 10 000 molecules. Under these conditions, each molecule of the collection was deposited, at a rate of approximately 0.2 μl of a 10 mM DMSO solution, by means of a 96-needle replicator, onto solid medium free of histidine and containing 1 mM of 3-AT. The robotic platform used for this screening was described previously by Beydon et al. (2000). Two molecules which are capable of restoring the yeast growth were thus isolated: for each of these two products, a strong white halo of restored growth can be readily distinguished around a growth inhibition zone related to a toxic effect of these molecules at high concentrations, whereas, for the control product 3, no effect is detected, as expected (FIG. 9).

In order to confirm the specificity of action of molecules isolated with this test, the effect of three other molecules that potentially inhibit the Fe65/App interaction (called A, B and C in FIG. 11) was tested on another type of protein-protein interaction using this interference system, the p53/mdm2 interaction (cf example 10.2).

First of all, the relative response of the UI interference system as a function of the Fe65/App and p53/mdm2 interactions was tested by means of a drop test. It was thus observed that the p53/mdm2 complex inhibits yeast growth on medium without histidine but at a concentration of 3-AT higher than that used with the strain containing the Fe65/App interaction (FIG. 10). This suggests that the p53/mdm2 interaction could be weaker than the Fe65/App interaction or else that the interactants are not as well expressed in the yeast.

The yRB49 strain was then transformed with the pair of plasmids pRB34 and pRB35, resulting in expression of the Fe65/App interaction, or the pair pDBT-mdm2 and pVP16-p53Nt, resulting in expression of the p53/mdm2 interaction.

The two Fe65/App and p53/mdm2 strains were each plated out onto dishes of medium free of histidine and supplemented, respectively, with 1 mM and 20 mM of 3-AT. For each of the three potentially inhibitory molecules, three drops of 1 μl of solutions at 0.1, 1 and 10 mM in DMSO were deposited per dish. In addition, 1 μl of histidine (0.125% w/v) was deposited at the center of another dish, as a positive control for growth restoration. A product taken randomly from the collection and used at the same concentrations as the three molecules tested was used as a negative control.

The results presented in FIG. 11 show that the products A and B are active, since they induce a halo of restored growth of the Fe65/App strain, the size of which is dependent on the concentration used. Such a concentration-dependent halo is not observed for the p53/mdm2 strain, which demonstrates the specificity of the inhibitory effect of these two molecules. On the other hand, the product C induces growth of the two strains, Fe65/App and p53/mdm2, and can therefore be categorized as active but nonspecific. As expected, the product D has no effect on the two strains tested; it is considered to be inactive.

The growth restoration test therefore makes it possible to isolate, from a library of several thousand molecules, molecules which are specific inhibitors of a protein/protein interaction.
All the publications and patents cited are incorporated in the present application by reference.

### BIBLIOGRAPHY

Alber, T., and Kawasaki, G. (1982). J Mol Appl Genet. 1, 419-434.
Bartel, P. L., C.-T. Chien, R. Sternglanz, and S. Fields. 1993. Using the two-hybrid system to detect protein-protein interactions., p. 153-179. In D. A. Hartley (ed.), Cellular interactions in development: a practical approach. Oxford University Press, Oxford.
Beydon, M-H., A. Fournier, L. Drugeault and J. Beyquard. (2000). J. Mol. Screen. 5: 12-21
Boeke, J. D., J. Trueheart, et al. 1987. Methods Enzymol 154: 164-75.
Bonneaud N, Ozier-Kalogeropoulos O, Li G Y, Labouesse M, Minvielle-Sebastia L, Lacroute F. 1991. Yeast. 7(6):609-15.
Brachmann, C. B., A. Davies, G. J. Cost, et al. 1998. Yeast 14:115-32.
Breeden, L. and K. Nasmyth. 1985. Regulation of the yeast HO gene. Cold Spring Harb Symp Quant Biol 50: 643-50.
Burke, T. W., J. G. Cook, M. Asano, and J. R. Nevins. 2001. J Biol Chem 276: 15397-408.
Cagney, G., P. Uetz, and S. Fields. 2000. Methods Enzymol 328:3-14.
Callen B P, Shearwin K E, Egan J B. 2004. Mol. Cell. 14(5):647-56.
Causier, B., and B. Davies. 2002. Plant Mol Biol 50:855-70.
Chem J, Marechal V, Levine A J. 1993. Mol Cell Biol. 13(7):4107-14.
Chevray P M and Nathans D. Proc Natl Acad Sci USA. 1992. 89(13):5789-93.
Chien, C. T., P. L. Bartel, R. Sternglanz, and S. Fields. 1991. Proc Natl Acad Sci USA 88:9578-82.
Coates, P. J., and P. A. Hall. 2003. J Pathol 199:4-7.
Crispino, J. D., M. B. Lodish, J. P. MacKay, and S. H. Orkin. 1999. Mol Cell 3:219-28.
Daros, J. A., M. C. Schaad, and J. C. Carrington. 1999. J Virol 73:8732-40.
Das, G. C., Niyogi, S. K., and Salzman, N. P. 1985. Prog Nucleic Acid Res Mol Biol 32: 217-236.
DeMarini, D. J., C. L. Creasy, et al. 2001. Biotechniques 30(3): 520-3.
Dormer, U. H., Westwater, J., McLaren, N. F., Kent, N. A., Mellor, J., and Jamieson, D. J. 2000. J Biol Chem 275: 32611-32616.
Elledge, S. J., and Spottswood, M. R. 1991. Embo J 10: 2653-2659.
Ernst, J. F. (1986). DNA 5, 483-491.
Ferreiro, J. A., Powell, N. G., Karabetsou, N., Kent, N. A., Mellor, J., and Waters, R. 2004. Nucleic Acids Res 32: 1617-1626.
Fields, S., and O, Song. 1989. Nature 340:245-6.
Fiore F, Zambrano N, Minopoli G, Donini V, Duilio A, Russo T. 1995. J Biol. Chem. 270(52):30853-6.
Foecking, M. K., and Hofstetter, H.1986. Gene 45: 101-105.
Gietz, R. D., and A. Sugino. 1988. Gene 74:527-34.
Gill, G. and M. Ptashne. 1987. Cell 51(1): 121-6.
Graham, I. R., and Chambers, A. 1996. Curr Genet. 30: 93-100
Greger, I. H., A. Aranda, and N. Proudfoot. 2000. Proc Natl Acad Sci USA 97:8415-20.
Greger, I. H., F. Demarchi, M. Giacca, and N. J. Proudfoot. 1998. Nucleic Acids Res 26:1294-301.
Greger, I. H., and N. J. Proudfoot. 1998. Embo J 17:4771-9.
Gutherie, C., and G. R. Fink (ed.). 2002. Guide to yeast genetics and molecular cell biology, vol. 350. Academic press, San Diego, Calif.
Gyuris, J., Golemis, E., Chertkov, H., and Brent, R. 1993. Cell 75, 791-803.
Harper, J. W., G. R. Adami, N. Wei, K. Keyomarsi, and S. J. Elledge. 1993. Cell 75:805-16.
Huang, J., and S. L. Schreiber. 1997. Proc Natl Acad Sci USA 94:13396-401.
Irniger, S., C. M. Egli, and G. H. Braus. 1991. Mol Cell Biol 11:3060-9.
Johnston, M., and R. W. Davis. 1984. Mol Cell Biol 4:1440-8.
Kaufer, N. F., H. M. Fried, W. F. Schwindinger, M. Jasin, and J. R. Warner. 1983. Nucleic Acids Res 11:3123-35.
Kronman, C., Velan, B., Gozes, Y., Leitner, M., Flashner, Y., Lazar, A., Marcus, D., Sery, T., Papier, Y., Grosfeld, H., and et al. 1992. Gene 121:295-304.
Leanna, C. A., and M. Hannink. 1996. Nucleic Acids Res 24:3341-7.
Longtine, M. S., A. McKenzie, 3rd, D. J. Demarini, N. G. Shah, A. Wach, A. Brachat, P. Philippsen, and J. R. Pringle. 1998. Yeast 14:953-61.
McLoughlin D M and Miller C C. 1996. FEBS Lett. 397(2-3):197-200.
Martens, J. A., Laprade, L., and Winston, F. 2004. Nature 429, 571-574.
Mizushima, S., and Nagata, S. 1990. Nucleic Acids Res 18: 5322.
Momand J, Zambetti G P, Olson D C, George D, Levine A J. 1992. Cell. 69(7):1237-45.
Nacken, V., Achstetter, T., and Degryse, E. (1996). Gene 175, 253-260.
Nagarajan, L., and Storms, R. K. 1997. J Biol Chem 272: 4444-4450.
Navarro, P., P. Durrens, and M. Aigle. 1997. Biochim Biophys Acta 1343:187-92.
Nenoi, M., Mita, K., Ichimura, S., Cartwright, I. L., Takahashi, E., Yamauchi, M., and Tsuji, H.1996. Gene 175: 179-185.
Oliner J D, Pietenpol J A, Thiagalingam S, Gyuris J, Kinzler K W, Vogelstein B. 1993. Nature. 362(6423):857-60.
Orr-Weaver, T. L. and J. W. Szostak. 1983. Proc Natl Acad Sci USA 80(14): 4417-21.
Osborne, B. I., and L. Guarente. 1989. Proc Natl Acad Sci USA 86:4097-101.
Pearlberg, J. 1995. Studies of the yeast transcription factor GAL11. Ph.D. thesis. Harvard University, Cambridge, Mass.
Peterson, J. A., and A. M. Myers. 1993. Nucleic Acids Res 21:5500-8.
Picard, D., Schena, M., and Yamamoto, K. R. 1990. Gene 86, 257-261.
Puig, S., J. E. Perez-Ortin, and E. Matallana. 1999. Curr Microbiol 39:369-0373.
Puthalakath, H., D. C. Huang, L. A. O'Reilly, S. M. King, and A. Strasser. 1999. Mol Cell 3:287-96.
Ren, B., Robert, F., Wyrick, J. J., Aparicio, O.,
Jennings, E. G., Simon, I., Zeitlinger, J., Schreiber, J., Hannett, N., Kanin, E., et al. 2000. Science 29: 2306-2309.
Ruohonen, L., M. K. Aalto, and S. Keranen. 1995. J Biotechnol 39:193-203.
Sambrook, J., E. F. Fritsch, and T. Maniatis. 1989. Molecular cloning: A laboratory manual. Cold Spring Habor Lab. Press, Plainview, N.Y.
Schorpp, M., Jager, R., Schellander, K., Schenkel, J., Wagner, E. F., Weiher, H., and Angel, P. 1996. Nucleic Acids Res 24: 1787-1788.
Serebriiskii, I., Khazak V. and Golemis, E. A. 1999. J Biol Chem 274, 17080-17087.
Serebriiskii, I. G., V. Khazak, and E. A. Golemis. 2001. Biotechniques 30:634-6, 638, 640 passim.
Sigalas, I., A. H. Calvert, et al. 1996. Nat Med 2(8): 912-7.
Sikorski, R. S., and Hieter, P. 1989. Genetics 122: 19-27.
Springer, C., O. Valerius, A. Strittmatter, and G. H. Braus. 1997. J Biol Chem 272:26318-24.
Steiner, S., and Philippsen, P. 1994. Mol Gen Genet 242, 263-271.
Svetlov, V. V., and Cooper, T. G. (1995). Yeast 11, 1439-1484.
Valerius, O., C. Brendel, K. Duvel, and G. H. Braus. 2002. J Biol Chem 277:21440-5.
Vidal, M., R. K. Brachmann, A. Fattaey, E. Harlow, and J. D. Boeke. 1996. Proc Natl Acad Sci USA 93:10315-20.
Vidal, M., and H. Endoh. 1999. Trends Biotechnol 17:374-81.
Vojtek, A. B., S. M. Hollenberg, et al. 1993. Cell 74(1): 205-14.
Vojtek, A. B. and S. M. Hollenberg 1995. Methods Enzymol 255: 331-42.
Wach A, Brachat A, Alberti-Segui C, Rebischung C, Philippsen P. 1997. Yeast. 13(11):1065-75.
Wahle, E., and U. Ruegsegger. 1999. FEMS Microbiol Rev 23:277-95.
Xu, C. W., Mendelsohn, A. R., and Brent, R. 1997. Proc Natl Acad Sci USA 94, 12473-12478.
Weinstock, K. G., and Strathern, J. N. 1993. Yeast 9, 351-361.
Winzeler E A, Shoemaker D D, Astromoff A et al. 1999. Science 285(5429), 901-6.
Yamamoto, T., de Crombrugghe, B., and Pastan, I. 1980. Cell 22: 787-797.
Yocum, R. R., Hanley, S., West, R., Jr., and Ptashne, M. 1984. Mol Cell Biol 4: 1985-1998.
Young, K., S. Lin, L. Sun, E. Lee, M. Modi, S. Hellings, M. Husbands, B. Ozenberger, and R. Franco. 1998. Nat Biotechnol 16:946-50.
Zambrano N, Buxbaum J D, Minopoli G et al. 1997. J Biol. Chem. 272(10):6399-405.
Zutshi, R., M. Brickner, and J. Chmielewski. 1998. Curr Opin Chem Biol 2:62-6.

1. A cell comprising an interference DNA construct wherein said construct comprises a reporter gene under control of a first promoter and at least one interference promoter wherein activation of the interference promoter involves a transcriptional interference of the first promoter leading to a detectable decrease in expression of the reporter genie, wherein said eel additionally express a first chimeric protein (Y-AD) comprising a transcription activation domain (AD) fused to a protein Y capable of interacting with a protein X, and a second chimeric protein (X-DBD) comprising a first DNA-binding domain (DBD) fused to a second domain comprising a protein X capable of interacting with the protein Y, wherein the interaction of the two chimeric proteins X-DBD and Y-AD leads to formation of a functional transcription factor activating, at least one of the interference promoter.

2. The cell as claimed in claim 1, characterized in that the first promoter regulating the expression of the reporter genie is an inducible promoter, the protein Y is capable of interacting with said protein X and a protein Z, and in that the cell expresses a third chimeric protein (Z-DBD) comprising second DNA-binding domain (DBD), fused to a third domain comprising the protein Z capable of interacting with the protein Y, the interaction of the two chimeric proteins Z-DBD and Y-AD leading to the formation of a functional transcription factor activating the expression of the reporter gene.

3. The cell as claimed in claim 2, characterized in that the inducible promoter regulating the expression of the reporter gene comprises a sequence capable of interacting with the DNA-binding domain (DBD) of the chimeric protein (Z-DBD).

4. The cell as claimed in claim 1, characterized in that the promoter regulating the expression of the reporter gene is a constitutive promoter.

5. The cell as claimed in claim 1, characterized in that the cell is a host cell transformed or transfected by at least one DNA construct coding for at least one of the interference promoters and at least one of the chimeric proteins, the whole of these constructs being carried by one or more nonintegrative vectors.

6. The cell as claimed in claim 5, characterized in that the cell is a host cell transformed or transfected by at least one of the DNA constructs the DNA constructs being carried by one or more nonintegrative vectors, and in that the DNA construct coding for at lest one of the interference promoters is integrated into the genome of the cell.

7. The cell as claimed in claim 5, characterized in that the DNA constructs coding for at least one of the interference promoters and at least one of the chimeric proteins are integrated into the genome of the cell.

8. The cell as claimed in claim 6 or 7, characterized in that the DNA construct coding for the at least one of interference promoters is integrated into a locus free of perturbatory genomic transcription activities.

9. The cell as claimed in claim 1 or 5, characterized in that the cell is elected from the group consisting of mammals, insects, plants and yeasts.

10. The cell as claimed in claim 9, characterized in that the cells are yeast cells.

11. The cell as claimed claim 10, characterized in that the yeast cells are selected from the group consisting of Saccharomyces cerevisiae, Schizosaccharomyces pombe, Kluyveromyces lactis, Pichia pastoris, Saccharomyces carlsbergensis and Candida albicans.

12. The cell as claimed in claim 1, characterized in that at least one interference promoter is positioned downstream of the reporter gene and of the first promoter and in an orientation opposite to the latter (DI).

13. The cell as claimed in claim 1, characterized in that at least one interference promoter is positioned downstream of the reporter gene and of the first promoter and in the same orientation as the latter (nDI).

14. The cell as claimed in claim 1, characterized in that at least one interference promoter is positioned upstream of the reporter gene and of the first promoter and in the same orientation as the latter (UI).

15. The cell as claimed in claim 1, characterized in that at least one interference promoter is positioned on both sides of the first promoter and of the reporter gene, the interference promoter(s) situated downstream of the first promoter, and of the reporter gene having a convergent orientation with respect to the first promoter and he interference promoter(s) positioned upstream of the first promoter and of the reporter gene having an orientation identical to that of the first promoter (UDI).

16. The cell as claimed in claim 1 characterized in that at least one interference promoter is positioned on both sides of the first promoter and of the reporter gene, the interference promoter(s) situated downstream of the first promoter and of the reporter gene having a paired orientation with respect to the first promoter and the interference promoter(s) positioned upstream of the first promoter and of the reporter gene having an orientation identical to that of the first promoter (nUDI).

17. The cell as claimed in claim 1, characterized in that the reporter gene is a gene essential to the survival of the cell.

18. The cell as claimed in claim 17, characterized in that the reporter gene is a gene indispensable to the primary metabolism, to cell division, to protein synthesis, to DNA synthesis or RNA synthesis.

19. The cell as claimed in claim 17, characterized in that the reporter gene is not in itself alone essential to the survival of the cell, but is essential to the survival of the coil when its transcription is inhibited in association with one or more reporter genes of the same type, the expression of which is or is not controlled by the transcriptional interference system.

20. The cell as claimed in claim 1, characterized in that the inducible interference promoter(s) comprise a sequence capable of interacting with the DNA-binding domain (DBD) of the chimeric protein (X-DBD).

21. The cell as claimed in claim 1, characterized in that the inducible interference promoter(s) comprise a sequence capable of interacting with a protein having a DNA-binding domain (DBD) selected from the group consisting of GAL4 UAS, LexAop, clop and TetRop and wherein the DNA-binding domain of the chimeric protein X-DBD is the corresponding DBD, respectively GAL4, LexA, cI or TetR.

22. The cell as claimed in claim 1, characterized in that the transcription activation domain (AD) of the chimeric protein Y-AD is selected from the group consisting of B42, VP16 and GAL4p.

23. The cell as claimed in claim 1, characterized in that the interference DNA construct is bordered at its ends by one or more unidirectional or bidirectional transcription terminators.

24. A method for identification of a compound inhibiting the interaction of a first protein X with a second protein Y, comprising the following steps:
a) culture cells as claimed in claim 1,
b) incubate said cells in the presence of the compound to be tested,
c) compare the expression of the reporter gene in the presence and in the absence of said compound, an increase in the expression of the reporter gene being the indication that the compound to be tested is an inhibitor of the interaction of the protein X with the partner protein Y expressed by the cultured cells.

25. The method of claim 24 for the identification of compounds inhibiting protein-protein interaction.

26. The method of claim 24 for the screening of cDNA banks or of banks of peptides in order to identify peptides or protein factors specifically abrogating a protein-protein interaction.

27. A kit for a double-hybrid system comprising:
A first DNA construct comprising:
A reporter gene placed under the control of a first promoter,
at least one inducible promoter, wherein activation involves transcriptional interference of the first promoter, leading to a detectable decrease in the expression of the reporter gene,
A second DNA construct coding for:
A first chimeric protein (Y-AD) comprising a transcription activation domain (AD) fused to a protein Y capable of interacting with a protein X,
A third DNA construct coding for:
A second chimeric protein (X-DBD) comprising a first DNA-binding domain (DBD) fused to a second domain formed by the protein X capable of interacting with the protein Y, wherein interaction of the the first chimeric protein and the second chimeric protein leads to formation of a functional transcription factor activating at least one interference promoter when the first chimeric protein and the second chimeric protein are expressed in a host cell.

28. The kit as claimed in claim 27, the first promoter regulating the expression of the reporter gene is an inducible promoter, wherein the protein Y is capable of interacting with protein X and protein Z, wherein said kit comprises a fourth DNA construct coding for a third chimeric protein (Z-DBD) comprising a DNA-binding domain (DBD), fused to a third domain formed by a protein Z capable of interacting with the protein Y, wherein interaction Z-DBD and Y-AD leads to the formation of a functional transcription factor activating expression of the reporter gene.

29. A method for identification of a compound inhibiting interaction of said protein X with said protein Y, but not inhibiting or inhibiting less the interaction between said protein Y and said protein Z, comprising the following steps:
a) culturing cells as claimed in claim 2,
b) incubating said cells in the presence of the compound to be tested, and
c) comparing expression of the reporter gene in presence and in absence of said compound, an increase in expression of the reporter gene indicating the compound is an inhibitor of the interaction of the protein X with the protein Y, but the compound does not inhibit or inhibits less the interaction between the protein Y and the protein Z.

30. A yeast integration vector comprising two fragments homologous to upstream and downstream regions of an open reading frame of gene URA3 of S. cerevisiae and allowing integration by homologous recombination at the level of the locus URA3 of a sequence inserted between the two fragments.


SEQUENCE LISTING (38 sequences)

SEQ ID NO: 1 - DNA, 37 bp, Artificial Sequence
  Oligonucleotide RB43
aaaatctagaggcgccatatccttttgttgtttccgg

SEQ ID NO: 2 - DNA, 35 bp, Artificial Sequence
  Oligonucleotide RB44
aaaaacgcgtggcgcccatctttcaggaggcttgc

SEQ ID NO: 3 - DNA, 27 bp, Artificial Sequence
  Oligonucleotide RB75
cgcgtggggggttaattaaaaaaaagc

SEQ ID NO: 4 - DNA, 27 bp, Artificial Sequence
  Oligonucleotide RB76
ggccgcttttttttaattaacccccca

SEQ ID NO: 5 - DNA, 29 bp, Artificial Sequence
  Oligonucleotide RB45
aaaaacgcgtacagagcagaaagccctag

SEQ ID NO: 6 - DNA, 52 bp, Artificial Sequence
  Oligonucleotide RB46
aaaaaagcggccgcggcgcgccttaattaactacataagaacacctttggtg

SEQ ID NO: 7 - DNA, 31 bp, Artificial Sequence
  Oligonucleotide RB51
aaaaggcgcgcctaattccgggcgaatttct

SEQ ID NO: 8 - DNA, 28 bp, Artificial Sequence
  Oligonucleotide RB52
aaaagagctctgcatgccggtagaggtg

SEQ ID NO: 9 - DNA, 93 bp, Artificial Sequence
  Oligonucleotide RB98
cgatcgcgtttgtacagaaaaaaaagaaaaatttgaaatataaataacgttcttaatactaacataactattaaaaaaaataaatagggaccg

SEQ ID NO: 10 - DNA, 95 bp, Artificial Sequence
  Oligonucleotide RB99
aattcggtccctatttattttttttaatagttatgttagtattaagaacgttatttatatttcaaatttttcttttttttctgtacaaacgcgat

SEQ ID NO: 11 - DNA, 34 bp, Artificial Sequence
  Oligonucleotide RB49
aaaaggcgcgccgtaaagagccccattatcttag

SEQ ID NO: 12 - DNA, 33 bp, Artificial Sequence
  Oligonucleotide RB50
aaaaggcgcgcctttgagatccgggttttttct

SEQ ID NO: 13 - DNA, 94 bp, Artificial Sequence
  Oligonucleotide RB81
ccggggcggccgcaacgtttggtggtggcgaccatcctccaaaatcggatctggttccgcgttgagtagctgaataagtgaataggcggccgct

SEQ ID NO: 14 - DNA, 94 bp, Artificial Sequence
  Oligonucleotide RB82
ctagagcggccgcctattcacttattcagctactcaacgcggaaccagatccgattttggaggatggtcgccaccaccaaacgttgcggccgcc

SEQ ID NO: 15 - DNA, 32 bp, Artificial Sequence
  Oligonucleotide RB55
aaaacccggggtaaagagccccattatcttag

SEQ ID NO: 16 - DNA, 33 bp, Artificial Sequence
  Oligonucleotide RB56
aaaacccgggcattttgagatccgggttttttc

SEQ ID NO: 17 - DNA, 34 bp, Artificial Sequence
  Oligonucleotide RB83
aaaaaagagctctactaaactcacaaattagagc

SEQ ID NO: 18 - DNA, 41 bp, Artificial Sequence
  Oligonucleotide RB84
aaaaaagaattcgcggccgcaaatatactggggaaccagtc

SEQ ID NO: 19 - DNA, 41 bp, Artificial Sequence
  Oligonucleotide RB85
aaaaaacgtacggcggccgcgataaggagaatccatacaag

SEQ ID NO: 20 - DNA, 32 bp, Artificial Sequence
  Oligonucleotide RB86
aaaaaacgtacgtttatggaccctgaaaccac

SEQ ID NO: 21 - DNA, 25 bp, Artificial Sequence
  Oligonucleotide RB38
ggggtaattaatcagcgaagcgatg

SEQ ID NO: 22 - DNA, 19 bp, Artificial Sequence
  Oligonucleotide RB61
cgatgtatgggtttggttg

SEQ ID NO: 23 - DNA, 19 bp, Artificial Sequence
  Oligonucleotide RB66
tcttgcgagatgatcccgc

SEQ ID NO: 24 - DNA, 22 bp, Artificial Sequence
  Oligonucleotide RB68
taagcgtattactgaaagttcc

SEQ ID NO: 25 - DNA, 23 bp, Artificial Sequence
  Oligonucleotide RB103
gcaatgaaagagcagagcgagag

SEQ ID NO: 26 - DNA, 25 bp, Artificial Sequence
  Oligonucleotide RB109
gttatcagatattatcaggtgggaa

SEQ ID NO: 27 - DNA, 33 bp, Artificial Sequence
  Oligonucleotide RB250
agatcgaattcaaggcggccggggaggccgagg

SEQ ID NO: 28 - DNA, 32 bp, Artificial Sequence
  Oligonucleotide RB251
gcaggtcgactcatggggtatgggcccccagc

SEQ ID NO: 29 - DNA, 55 bp, Artificial Sequence
  Oligonucleotide RB136
ttgtaaaacgacggccagtgaattccgtacgatatccttttgttgtttccgggtg

SEQ ID NO: 30 - DNA, 24 bp, Artificial Sequence
  Oligonucleotide RB137
agttgattgtatgcttggtatagc

SEQ ID NO: 31 - DNA, 9728 bp, Artificial Sequence
  Vector pRB34
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccgtacgatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagctttgcaaagatggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatcgaattcaaggcggccggggaggccgaggaggaggaggaggatgatgatgatgaagaggaggaggaggacttatcttctcccccagggctgcctgagcccctggagagtgtggaggcccctcccaggccccaagcccttacagatggcccccgggaacacagcaagagtgccagcctcctgtttggcatgcggaacagtgcagccagtgatgaggactcaagctgggctaccttatcccagggcagcccctcctatggctccccagaggacacagattccttctggaaccccaacgccttcgagacggattccgacctgccggctggatggatgagggtccaggacacctcagggacctattactggcacatcccaacagggaccacccagtgggaaccccccggccgggcctccccctcacaggggagcagcccccaagaggagtcccagctcacctggacaggttttgctcatggagaaggctttgaggatggagaattttggaaggatgaacccagtgatgaggccccaatggagctgggactgaaggaacctgaggaggggacgttgaccttcccagctcagagcctcagcccagagccgttgccccaagaggaggagaagcttcccccacggaataccaacccagggatcaagtgtttcgccgtgcgctccctaggctgggtagagatgaccgaggaggagctggcccctggacgcagcagtgtggcagtcaacaattgcatccgtcagctctcttaccacaaaaacaacctgcatgaccccatgtctgggggctggggggaaggaaaggatctgctactgcagctggaggatgagacactaaagctagtggagccacagagccaggcactgctgcacgcccaacccatcatcagcatccgcgtgtggggcgtcgggcgggacagtggaagggactttgcctacgtagctcgtgataagctgacccagatgctcaagtgccacgtgtttcgctgtgaggcacctgccaagaacatcgccaccagcctgcatgagatctgctctaagatcatggccgaacggcgtaatgcccgctgcttggtaaatggactctccctggaccactctaaacttgtggatgtccctttccaagtggaattcccagcgcctaagaatgagttggtccagaagttccaagtctattacctggggaatgtacctgttgctaaacctgttggggtagatgtgattaatggggccctcgagtcagtcctgtcctccagcagccgtgaacaatggaccccaagtcatgtcagtgtggcccctgctaccctcaccatcttgcaccagcagacagaggcagtgctgggagagtgtcgggtgcgtttcctctccttcctggccgtgggcagagatgtccacacgtttgcattcatcatggctgccggcccagcctccttctgctgccacatgttctggtgcgagcccaatgctgccagcctctcagaggctgtgcaggctgcgtgcatgcttcgctaccagaagtgtctggatgcccgttcccaggcctccacctcctgcctcccagcaccccctgctgagtctgtggcacggcgtgtagggtggactgtccgcaggggtgttcagtcgctgtggggctccctgaagcccaaacggctgggggcccataccccatgagtcgacctgcagagatctatgaatcgtagatactgaaaaaccccgcaagttcacttcaactgtgcatcgtgcaccatctcaatttctttcatttatacatcgttttgccttcttttatgtaactatactcctctaagtttcaatcttggccatgtaacctctgatctatagaattttttaaatgactagaattaatgcccatcttttttttggacctaaattcttcatgaaaatatattacgagggcttattcagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcagatctattacattatgggtggtatgttggaataaaaatcaactatcatctactaactagtatttacgttactagtatattatcatatacggtgttagaagatgacgcaaatgatgagaaatagtcatctaaattagtggaagctgaaacgcaaggattgataatgtaataggatcaatgaatattaacatataaaatgatgataataatatttatagaattgtgtagaattgcagattcccttttatggattcctaaatcctcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagccgcggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccacggaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgtagtcgacctcctcttcagtactaaatctaccaataccaaatctgatggaagaatgggctaatgcatcatccttacccagcgcatgtaaaacataagaaggttctagggaagcagatgtacaggctgaacccgaggataatgcgatatcccttagtgccatcaataaagattctccttccacgtaggcgaaagaaacgttaacacaccctggataacgatgatctggagatccgttcaacgtggtatgttcagcggataatagacctttgactaatttatcggatagtcttttgatgtgagcttggtcgttgtcaaattctttcttcatcaatctcgcagcttcaccaaatcccgctaccaatgggggggccaaagtaccagatctgctgcattaatgaatcggccaacgcgcggggagaggcggtttgcgtattgggcgctcttccgcttcctcgctcactgactcgctgcgctcggtcgttcggctgcggcgagcggtatcagcatcgatgaatggaaattacgtactttcaacgcaagagcaagacaaaagaaagcctaaaactgtcttttcgtgtcggtcgtccaatatcatcgtaaacgtgtgtagagccttaaattctaatttctaactttaaattttttttgctattataatactaatttctaactttcaacctattttacatcttcggaaaacaaaaactattttttctttaatttctttttttactttctatttttaatttatatatttatattaaaaaatttaaattatattatttttatagcacgtgatgaaaagaaatatagtcttcaaggttatattatacaggtttatatattattttacttatttttgttgttctttttcttggagtaatttagcattaacaacttcgacccatcgcgatgacgaaattgcacgtttaaaggagaagtcggaatcgttgcaagaagaaataaattagtttgacgttttattccttttcttctgcacagaagtgtgtatatatatatttatatatagtcgatgtgcaaaagcctacattatttttcttttgatttttatttttttgttcaagtaggttcatgccgacagtaaatccttatgccctacctaaatataaacacccaaatatatatatatatatatatatatataaataataaaaacggaaaggaaatagaataataataataatactacacgtttacgtatgtaactcattctattattcagactttgatattaatagaattattactactaggtttcctcacttcaaaatctataaatataccgtcatgctctaatcgatgctcactcaaaggtcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 32 - DNA, 147 bp, Artificial Sequence
  Oligonucleotide RB252
aattcaagaaacagtacacatccattcatcatggtgtggtggaggttgacgccgctgtcaccccagaggagcgccacctgtccaagatgcagcagaacggctacgaaaatccaacctacaagttctttgagcagatgcagaactagg

SEQ ID NO: 33 - DNA, 147 bp, Artificial Sequence
  Oligonucleotide RB253
tcgacctagttctgcatctgctcaaagaacttgtaggttggattttcgtagccgttctgctgcatcttggacaggtggcgctcctctggggtgacagcggcgtcaacctccaccacaccatgatgaatggatgtgtactgtttcttg

SEQ ID NO: 34 - DNA, 5879 bp, Artificial Sequence
  Vector pRB35
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcccggaattagcttggctgcaggtcgacctagttctgcatctgctcaaagaacttgtaggttggattttcgtagccgttctgctgcatcttggacaggtggcgctcctctggggtgacagcggcgtcaacctccaccacaccatgatgaatggatgtgtactgtttcttgaattccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatctttcaggaggcttgcttcaagcttggagttgattgtatgcttggtatagcttgaaatattgtgcagaaaaagaaacaaggaagaaagggaacgagaacaatgacgaggaaacaaaagattaataattgcaggtctatttatacttgatagcaagacagcaaacttttttttatttcaaattcaagtaactggaaggaaggccgtataccgttgctcattagagagtagtgtgcgtgaatgaaggaaggaaaaagtttcgtgtgcttcgagatacccctcatcagctctggaacaacgacatctgttggtgctgtctttgtcgttaattttttcctttagtgtcttccatcatttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaagaggaagtccatattgtacacccggaaacaacaaaaggatatcgtacgcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcagatctgggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaaactttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacggtatatatactcacgtgctcaatagtcaccaatgccctccctcttggccctctccttttcttttttcgaccgaattaattcagatctgctgcattaatgaatcggccaacgcgcggggagaggcggtttgcgtattgggcgctcttccgcttcctcgctcactgactcgctgcgctcggtcgttcggctgcggcgagcggtatcagcatcgatgaatggaaattacgtactttcaacgcaagagcaagacaaaagaaagcctaaaactgtcttttcgtgtcggtcgtccaatatcatcgtaaacgtgtgtagagccttaaattctaatttctaactttaaattttttttgctattataatactaatttctaactttcaacctattttacatcttcggaaaacaaaaactattttttctttaatttctttttttactttctatttttaatttatatatttatattaaaaaatttaaattatattatttttatagcacgtgatgaaaagaaatatagtcttcaaggttatattatacaggtttatatattattttacttatttttgttgttctttttcttggagtaatttagcattaacaacttcgacccatcgcgatgacgaaattgcacgtttaaaggagaagtcggaatcgttgcaagaagaaataaattagtttgacgttttattccttttcttctgcacagaagtgtgtatatatatatttatatatagtcgatgtgcaaaagcctacattatttttcttttgatttttatttttttgttcaagtaggttcatgccgacagtaaatccttatgccctacctaaatataaacacccaaatatatatatatatatatatatatataaataataaaaacggaaaggaaatagaataataataataatactacacgtttacgtatgtaactcattctattattcagactttgatattaatagaattattactactaggtttcctcacttcaaaatctataaatataccgtcatgctctaatcgatgctcactcaaaggtcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 35 - DNA, 174 bp, Artificial Sequence
  Oligonucleotide RB200
ggccgcagtgaaccattgttcaatatcgtccggggacagcatcaaatcatccattgcttgggacggcaagggggacagaacgttgttttcaggaagtagtttccataggtctgaaaatgtttcctgactcagagggggctcgacgctaggatctgactgcggctcctccatctg

SEQ ID NO: 36 - DNA, 174 bp, Artificial Sequence
  Oligonucleotide RB201
gatccagatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgc

SEQ ID NO: 37 - DNA, 44 bp, Artificial Sequence
  Oligonucleotide RB202
cccgggaattcagatccatatgtgcaataccaacatgtctgtac

SEQ ID NO: 38 - DNA, 35 bp, Artificial Sequence
  Oligonucleotide RB203
acttagagctctaggggaaataagttagcacaatc